SEQUENCE LISTING (41 sequences)

SEQ ID NO: 1 - DNA, 2436 bp, Glycine max
  source 1..2436: mol_type = genomic DNA
gggggcggggggagataagagagagagagagagtggcagaggaactgatagagaactttcaaagctctttatcttctaccatcactcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtcctcgaagaccatacctcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 2 - DNA, 23 bp, synthetic construct
  source 1..23: mol_type = other DNA
ccggatgaagaggtatggtcttc

SEQ ID NO: 3 - DNA, 25 bp, synthetic construct
  source 1..25: mol_type = other DNA
tggcaggatatattgtggtgtaaac

SEQ ID NO: 4 - DNA, 26 bp, synthetic construct
  source 1..26: mol_type = other DNA
gtaaacctaagagaaaagagcgttta

SEQ ID NO: 5 - DNA, 63 bp, Glycine max
  source 1..63: mol_type = genomic DNA
gtactctagagatggcaaacaagtcctcgaagaccatacctcttcatccggtaaacttcatga

SEQ ID NO: 6 - protein, 17 aa, Glycine max
YSRDGKQVLEDHTSSSG

SEQ ID NO: 7 - DNA, 58 bp, synthetic construct
  source 1..58: mol_type = other DNA
gtactctagagatggcaaacaagtcctcgaagacctcttcatccggtaaacttcatga

SEQ ID NO: 8 - DNA, 56 bp, synthetic construct
  source 1..56: mol_type = other DNA
gtactctagagatggcaaacaagtcctcgaacctcttcatccggtaaacttcatga

SEQ ID NO: 9 - DNA, 55 bp, synthetic construct
  source 1..55: mol_type = other DNA
gtactctagagatggcaaacaagtcctcgacctcttcatccggtaaacttcatga

SEQ ID NO: 10 - protein, 255 aa, Glycine max
MRPERNPLDLNNLPDEYSRDGKQVLEDHTSSSGCRKKKSGGKDGKDECGKVYECRFCSLKFCKSQALGGHMNRHRQERETETLNQARQLVFRCDHNIAAQGAPHLGCCQTIGTGGYHPSGDPTVPLRFPRYFSGSSSTHMPPSPPPPPPPQRPYLYPSPTRPVSFGSSHFPLQHAVNDYYVGHVMSGGSHGHYVGGESTRSYTCIGAPVGQGGGFAGGKEGSAVQEEGLSTWGRGYSGAQDRLDPPSAINRFQDG

SEQ ID NO: 11 - DNA, 771 bp, Glycine max
  source 1..771: mol_type = other DNA
atgagaccagaacgaaaccccttagatcttaacaatttgcccgatgagtactctagagatggcaaacaagtcctcgaagaccatacctcttcatccggttgcaggaaaaagaaaagcggcgggaaggatggaaaagacgagtgtgggaaggtctacgagtgtagattttgttccctcaagttctgcaagtctcaggctcttgggggacacatgaaccgccaccgccaagagagggaaacggagacgctgaaccaggctcgtcaactggtctttcgttgtgatcataacattgctgcacaaggtgcccctcacttaggatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaa

SEQ ID NO: 12 - protein, 52 aa, synthetic construct
MRPERNPLDLNNLPDEYSRDGKQVLEDLFIRLQEKEKRREGWKRRVWEGLRV

SEQ ID NO: 13 - protein, 68 aa, synthetic construct
MRPERNPLDLNNLPDEYSRDGKQVLEPLHPVAGKRKAAGRMEKTSVGRSTSVDFVPSSSASLRLLGDT

SEQ ID NO: 14 - protein, 51 aa, synthetic construct
MRPERNPLDLNNLPDEYSRDGKQVLDLFIRLQEKEKRREGWKRRVWEGLRV

SEQ ID NO: 15 - DNA, 2431 bp, synthetic construct
  source 1..2431: mol_type = other DNA
gggggcggggggagataagagagagagagagagtggcagaggaactgatagagaactttcaaagctctttatcttctaccatcactcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtcctcgaagacctcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 16 - DNA, 766 bp, synthetic construct
  source 1..766: mol_type = other DNA
atgagaccagaacgaaaccccttagatcttaacaatttgcccgatgagtactctagagatggcaaacaagtcctcgaagacctcttcatccggttgcaggaaaaagaaaagcggcgggaaggatggaaaagacgagtgtgggaaggtctacgagtgtagattttgttccctcaagttctgcaagtctcaggctcttgggggacacatgaaccgccaccgccaagagagggaaacggagacgctgaaccaggctcgtcaactggtctttcgttgtgatcataacattgctgcacaaggtgcccctcacttaggatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaa

SEQ ID NO: 17 - DNA, 2429 bp, synthetic construct
  source 1..2429: mol_type = other DNA
gggggcggggggagataagagagagagagagagtggcagaggaactgatagagaactttcaaagctctttatcttctaccatcactcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtcctcgaacctcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 18 - DNA, 764 bp, synthetic construct
  source 1..764: mol_type = other DNA
atgagaccagaacgaaaccccttagatcttaacaatttgcccgatgagtactctagagatggcaaacaagtcctcgaacctcttcatccggttgcaggaaaaagaaaagcggcgggaaggatggaaaagacgagtgtgggaaggtctacgagtgtagattttgttccctcaagttctgcaagtctcaggctcttgggggacacatgaaccgccaccgccaagagagggaaacggagacgctgaaccaggctcgtcaactggtctttcgttgtgatcataacattgctgcacaaggtgcccctcacttaggatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaa

SEQ ID NO: 19 - DNA, 2428 bp, synthetic construct
  source 1..2428: mol_type = other DNA
gggggcggggggagataagagagagagagagagtggcagaggaactgatagagaactttcaaagctctttatcttctaccatcactcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtcctcgacctcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 20 - DNA, 763 bp, synthetic construct
  source 1..763: mol_type = other DNA
atgagaccagaacgaaaccccttagatcttaacaatttgcccgatgagtactctagagatggcaaacaagtcctcgacctcttcatccggttgcaggaaaaagaaaagcggcgggaaggatggaaaagacgagtgtgggaaggtctacgagtgtagattttgttccctcaagttctgcaagtctcaggctcttgggggacacatgaaccgccaccgccaagagagggaaacggagacgctgaaccaggctcgtcaactggtctttcgttgtgatcataacattgctgcacaaggtgcccctcacttaggatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaa

SEQ ID NO: 21 - DNA, 91 bp, synthetic construct
  source 1..91: mol_type = other DNA
tactctagagatggcaaacaagtcctcgatcttcatccggtaaacttcatgatcataccaatatatatatatatgcacgctgaaatatcaa

SEQ ID NO: 22 - protein, 67 aa, synthetic construct
MRPERNPLDLNNLPDEYSRDGKQVLDLHPVAGKRKAAGRMEKTSVGRSTSVDFVPSSSASLRLLGDT

SEQ ID NO: 23 - DNA, 96 bp, synthetic construct
  source 1..96: mol_type = other DNA
tactctagagatggcaaacaagtcctcgaatacctcttcatccggtaaacttcatgatcataccaatatatatatatatgcacgctgaaatatcaa

SEQ ID NO: 24 - protein, 52 aa, synthetic construct
MRPERNPLDLNNLPDEYSRDGKQVLEYLFIRLQEKEKRREGWKRRVWEGLRV

SEQ ID NO: 25 - DNA, 100 bp, synthetic construct
  source 1..100: mol_type = other DNA
tactctagagatggcaaacaagtcctcgaagacatacctcttcatccggtaaacttcatgatcataccaatatatatatatatgcacgctgaaatatcaa

SEQ ID NO: 26 - protein, 70 aa, synthetic construct
MRPERNPLDLNNLPDEYSRDGKQVLEDIPLHPVAGKRKAAGRMEKTSVGRSTSVDFVPSSSASLRLLGDT

SEQ ID NO: 27 - DNA, 97 bp, synthetic construct
  source 1..97: mol_type = other DNA
tactctagagatggcaaacaagtcctcgaagtacctcttcatccggtaaacttcatgatcataccaatatatatatatatgcacgctgaaatatcaa

SEQ ID NO: 28 - protein, 69 aa, synthetic construct
MRPERNPLDLNNLPDEYSRDGKQVLEVPLHPVAGKRKAAGRMEKTSVGRSTSVDFVPSSSASLRLLGDT

SEQ ID NO: 29 - DNA, 94 bp, synthetic construct
  source 1..94: mol_type = other DNA
tactctagagatggcaaacaagtcctcgaagctcttcatccggtaaacttcatgatcataccaatatatatatatatgcacgctgaaatatcaa

SEQ ID NO: 30 - protein, 68 aa, synthetic construct
MRPERNPLDLNNLPDEYSRDGKQVLEALHPVAGKRKAAGRMEKTSVGRSTSVDFVPSSSASLRLLGDT

SEQ ID NO: 31 - DNA, 2426 bp, synthetic construct
  source 1..2426: mol_type = other DNA
gggggcggggggagataagagagagagagagagtggcagaggaactgatagagaactttcaaagctctttatcttctaccatcactcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtcctcgatcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 32 - DNA, 2431 bp, synthetic construct
  source 1..2431: mol_type = other DNA
gggggcggggggagataagagagagagagagagtggcagaggaactgatagagaactttcaaagctctttatcttctaccatcactcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtcctcgaatacctcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 33 - DNA, 2435 bp, synthetic construct
  source 1..2435: mol_type = other DNA
gggggcggggggagataagagagagagagagagtggcagaggaactgatagagaactttcaaagctctttatcttctaccatcactcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtcctcgaagacatacctcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 34 - DNA, 2432 bp, synthetic construct
  source 1..2432: mol_type = other DNA
gggggcggggggagataagagagagagagagagtggcagaggaactgatagagaactttcaaagctctttatcttctaccatcactcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtcctcgaagtacctcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 35 - DNA, 2429 bp, synthetic construct
  source 1..2429: mol_type = other DNA
gggggcggggggagataagagagagagagagagtggcagaggaactgatagagaactttcaaagctctttatcttctaccatcactcaaccagcctctatattgcagtctcaaactgaaagtctaaatttttttgtaaaaagctttagttttatccctacccccaccccatctgaaagaaagagtgtttgcctcacatcatttttcccctttctgtctctctctctgtcggtaccatgtaggtcttcccccactactacaccttcacacccttcttttcttcctcctctttctccttctctaaaccctttaactttctctctcttatgactttgttgttcctttacaggagaccagaacgaaaccccttagatcttaacaatttgcccgatgagtactctagagatggcaaacaagtcctcgaagctcttcatccggtaaacttcatgatcataccaatatatatatatatgcacgctgaaatatcaaaggaacactttttttttctttctatttaccgaattctatttcacaatcacacaccaccatgggaagcctttcacttttcacaggaaacaaagttgaacactgcccgtggtttgttacccatccctgtggtatattccttgaccatgaaacagtgttcaccacacttttttttcctcatttttttattctttttgttttgtccttgtatacgtttcttttctttttttgtttcttctccttttcctcttctacttcgttatagtatctcttaaaagttatggaaaaaataaaataagaaaaacataaaagtagttaggtgatagaatagaacttaaaaaaaagaaaagtagttgtttgggggcgagcggtaagttaattaattgaattgatttgatggaggatttgggtaatcgagaatggggagttaaaggcaaagaagggtgtcatggaagttgctttaattggatggttatagttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtaccacctatattattatactttatattttcttcctcttttcttttaccactccttctcacaagaaccaaatctcttctggccgttaatgatttggccatgaaagcttgtcatttcaatcacaaaatgttgtgttgttggattgctgaaattcgatcatcgagagggccccttcagctgttttttttttttctttttgctttgctttattatttgtttttgtgattgttttgagcagagagggaaacggagacgctgaaccaggctcgtcaactggtctttcgttgtgatcataacattgctgcacaaggtgcccctcacttagggtatgcacccattacttccatgcagcgattctcttcctatttcttcttcttgttcaacatttatatatttcatttctcaaatacttgtttttctgtgggttctttagcgtttgttaacgttgttttttcaaggtataacacataatatttgggtactcagcatgacactgtttgatactgcgatttatttgtgataatattcaattcagatgctgccaaacaataggaacggggggttatcatccctcaggagacccaacagtgcctctaagattcccaagatacttctcaggttcatcctcaactcacatgccaccatccccgccaccgccgccgccaccgcaacgaccatacctatacccttcacctacgaggccagtgtcatttgggtcatcacacttccctctccagcatgcagtgaacgattactatgtgggccacgtgatgagtggtggcagccacggacactatgttggaggagagagcacaaggagttacacgtgcattggtgcaccggtggggcaaggtggcggattcgctggtggtaaggaggggtctgcagtgcaggaggaagggttgagtacttggggaaggggctattcaggtgcacaggatcgtttggatcctccctcagcgatcaatcggtttcaagatggtttctaaagagatgagagattctttgtttgagtggtttttggtttgtgttatgtttctgttatgttatgtggtatcatcataaagacacgcaatccagagagagagagaggtggtttttggtttatctacagtaaccagggacagcttttgagctcatggacacgctcagctactttggcttggagttgtgggtgggatttcttctcaacatcgccttttgtattggtagctagctagctagcttggacaaccagttagatttgaactgaaactttccaaactccttttcctttgcatgccaaattacaaccattttccttctcagtaatttgttctttcaa

SEQ ID NO: 36 - DNA, 4725 bp, Glycine max
  source 1..4725: mol_type = genomic DNA
taatcaataacccccaccactattttttttttaattttttttaaaatgtaaaacagttaattgcttctgcatattattaatttggacaacattaattttacctagctaactagttaactatcttataaaggagtcactgatcactagagatggagctagtactgtttggattcaaagaaatgcatctaaagcgaacagagaatataatgatgaatgtgagacatacattaagaaaattcaaatccatatatgtctccagttcatgcacatccattgaaaaggactatgtatatgtaatctatgagatatagcaactttcaaagaataaaacaacaagtgggggtcgagtctatctcaacttagattgctaaaaaccctaatgatagatactgatagcaacccttccttgatgcaagtctaagtcccctgggtagaattttacacccagaaggaggcattgagctactgtggaccatacatgacctgttctcaaacatgccccccctctttaagcacaataaccacgcaaaatgtctacgtaccctactccttgtcttgctttctttctggtataaatgtcatctttcattcctagggcaaactttaatgctcaaagatttgtactgtacatttgatttccatcctttgttggtaaagattttcatttggaatattaattttttttttctcaatgcatttgactgatattatttgaatattttttgaccctttaatttgttgatagctctagctagggcaaaacaacaaaaaacaagaaaactagatgcagaagtatatcatataattatgcaagatcatatttattaataaaattatataataaccagtataatgctcatttttttctaccttcatcatataattcactttatcgcacatttctttttatctcttatttatatatttattaaattaatataatttctgtatatattaggaataattaatacaaaagcaacatgtttcagaatcacatatctttgcactttataattctgtctgtcatttcccaagtctcaaccaaaccagacgaagctagctagcaatttgcaaaaaaactgattaagttaagccccccacttccactaccacactcacgctctctttttaaagtcacgagacccactgtcccattgttcattcaatttttggtgtggggggagataagagagagagagagagagtggcagaggaaccgatagggaactttcaaagctctttatcttctaccatcactcaactagcctctatattgcagtctcaaactgaaagtctcacaaattttgtagaaaactgtagttttatccccacccccctcccccatctgaaagaaagagtatttgcctcgcatcatttttcttttctttctctctttctgtctctctctctgtcggtaccatgtaggtcttcccccactactacaccttcacacccttcttctcatcctcctctttctccttctctaactctaagcccttttaactttctctctatctttttctctctcttatgactttgtcgttcctttacaggagaccagaacgaaacccattagatcttaacaatttgcccgatgagtactctagggatggcaaacaagtcctcgaagaccatacctcttcacccggtaaacttcatgatcatatctatctatctatctatatatatatatagaagcacgctagaatatcaaaggaatactttttttctttatatattccgaattctatttcacaaacacacaccatgggaagcctttcacttttcacaggaaacaaagttgaacactgccgtggtttgttacttacctctgtggtatattccttgaccatgaaacagtgttcaccacacttctttttttattcttttggttttgtccttgtatacgtttccttctctcctttttctttttttcctcttcttctcttaaaagataaaagaaaaggaaaaaaaagaagaagcataaaagtagttaggtgatagaacttaaaataaagaaaagtaattgttggggcgagcgattagttaattaattaaaatgaattgaattgatttgagggatttgggtaatcgagaatgaggcgtgaaagggaaagaaggtggtgtcatggaagttgctgtaattggatggttatggttgcaggttgcaggaaaaagaaaagcggcgggaaggatggaaaagacgagtgtgggaaggtctacgagtgtagattttgttccctcaagttctgcaagtctcaggctcttgggggacacatgaaccgccaccgccaaggctagtatataccacctattatatcttttcgtttttcttcctctttactctcacaagaaccaaatcttcactgcccgttaatgatttggccatgaagggttgtcatttcaatcaaaaaaagttgagttagctgaaattcgatccagagggtcccttcagctgtttttctttttttctttttttaattctttctttctttttgctttgcttttattattttgttttgagcagagagggaaacggagacgctgaaccaggctcgtcaactggtatttcgtagcgatcatatcattgctccacaaggtgcccctcacttagggtatgcacccattacttccatgcagctattctcttccaatttgttgttgttgttccaccctaatattacactatcattcccagttctttcttaaatacttgtttttctctaggttctttagcatttgttaacgttgttttttcaagttaacgcataatattagggtgcgcagcatgacactgtttgatacaattgactgtgataatattcagatgctgccagccaataggaacggggggttatcacccatcaggagacccaacagtgcctctaagattcccgagatacttctcaggttcatcctcaactcacatgccaccaccgccgccgccgccgccgccaccgcaacaatcacacctatacgcatcaccttcgaggccagtgtcatttgggtcatcacacttcccccaccagcatgctgtgaacgattactatgtgggccacgtgatgagtggtgggagccacggacactatgttggaggagagagcacgagtagttacacgtacattggtgccccggtggggcaagctggtggattcgctggtggtggtaaggaggggtcagcagtgcaggaggaagggttgagttggggaaggagctattcaggaggagcacagcatcgtttggatcctccctcagcgatcaatcggtttcatgatggtttctaatgagatgagatgagatgagagattcttttgtttgagtgtttttggtttgcgttttgtgttatgttattttatgtggtatcatcataaagacacgcaatccagagagagagacataacaagctgacacatggggtcatggagaggagggtatagctaaggctaggatgaagagagagagtgtgcgtgagaggtggtttttggtttatctacagtaaccaggaacagcttttgagctcatggacacgctcagctactttggcttggagttatgggtgggatttcttctcaacatcgccttttgtattggtagctacctatcttggacaaccagttagatatgaactgaaactttccaaactccttttgctttgcatgccaaattacaaccattttccttctaatttgcttctttcaacatactactattactattttatttatatatatctatttatgtgtgcatgtctgtttttgtgtttaatttaccatttcaaattccctaaagccccccgtcactctcgatcgctctcactctttctatccaaaccaaaattgggatctactaatttcttgaaatgttttatggtctcagtgtcacactatgctagcacaattagggtaatgacgcagagaagttttggagtcctcttacgaaaaaactatctagctgaattaatgctccctgaataggatattcaaacaatcaaacctgcaaaatatctaatttcttcttaaagaaaagctttatcctccctcactagctatttccacgatattgtaaataattttagaatacagagattatttattgcagcagacagtgaatgcagctatatttaatttgtttggagcataccaaacaaaactactatctaaggaataaaaacactcttgctgtcagctccgagagcttctaccaaatgcagcagctaagagaaatatatacacagccttaagtagctcatgatcgttcttgaaacagtaaaacacgtgtaaataatgtaaccacattattattagcatgatattttctcttgttttcaatacttgttggaaatgaatatgtttttctttgtatataggttgtacgtactccctttacgcacagtaattgaggatataattataagaaaaattatcctcttaaaaattattttaaaagaaaaagtgtttttctggcttcttgtcctatatatatatatattagttcgtgctatatattttagttcgttttttcactcaaggagaattaaatattccttcactcaacaaaggataatatgttaaaccgtttataagtgaagtttatttttttctaggcatggtgaaaatggtttgtatgaaaaggaagggtgatggttagtgacttatatatggcagtgttgatagtgtttatggtacatccaaatatccaatggcaatagcaacattggctgatgcg

SEQ ID NO: 37 - DNA, 774 bp, Glycine max
  source 1..774: mol_type = other DNA
atgagaccagaacgaaacccattagatcttaacaatttgcccgatgagtactctagggatggcaaacaagtcctcgaagaccatacctcttcacccggttgcaggaaaaagaaaagcggcgggaaggatggaaaagacgagtgtgggaaggtctacgagtgtagattttgttccctcaagttctgcaagtctcaggctcttgggggacacatgaaccgccaccgccaagagagggaaacggagacgctgaaccaggctcgtcaactggtatttcgtagcgatcatatcattgctccacaaggtgcccctcacttaggatgctgccagccaataggaacggggggttatcacccatcaggagacccaacagtgcctctaagattcccgagatacttctcaggttcatcctcaactcacatgccaccaccgccgccgccgccgccgccaccgcaacaatcacacctatacgcatcaccttcgaggccagtgtcatttgggtcatcacacttcccccaccagcatgctgtgaacgattactatgtgggccacgtgatgagtggtgggagccacggacactatgttggaggagagagcacgagtagttacacgtacattggtgccccggtggggcaagctggtggattcgctggtggtggtaaggaggggtcagcagtgcaggaggaagggttgagttggggaaggagctattcaggaggagcacagcatcgtttggatcctccctcagcgatcaatcggtttcatgatggtttctaa

SEQ ID NO: 38 - protein, 257 aa, Glycine max
MRPERNPLDLNNLPDEYSRDGKQVLEDHTSSPGCRKKKSGGKDGKDECGKVYECRFCSLKFCKSQALGGHMNRHRQERETETLNQARQLVFRSDHIIAPQGAPHLGCCQPIGTGGYHPSGDPTVPLRFPRYFSGSSSTHMPPPPPPPPPPQQSHLYASPSRPVSFGSSHFPHQHAVNDYYVGHVMSGGSHGHYVGGESTSSYTYIGAPVGQAGGFAGGGKEGSAVQEEGLSWGRSYSGGAQHRLDPPSAINRFHDGF

SEQ ID NO: 39 - DNA, 2819 bp, Glycine max
  source 1..2819: mol_type = genomic DNA
ggggagggaggacccacacacacaggattacacatgatgctttttttgacctgatcgagctgatgggagagaaaaaactgggaaattttatatttttaaatattttttacaaattttttcagaactaggatgggatagaaataaataaaaaaagtacacaataagagttgctaatagaataaaaaaaattctttattggatgagaaatatccatgcatccggtgtacagccaatcaattaagaaccgattccaagggccccattctgtgtttatgaccagcggagagagaaagagagtggaaaagtggaaaggataactcgtcacatatcatgactaatgtgatttcagcacaaacatcccagacccaaaggcaattgttgagatgagccgaattaagagacaatatattagtagtagtgggaggacctgcacggatgcacttgatttatttctagccttttttcattttttgtttttcctatagaacttgtattttctctagctaaatctaaattatgtattgcaacatattttaaaggaggggcaaaacacactcgatctaccttcaaagaaacttttaagacattataaatatggagcccacactcccttcttcctcagtaagatcatcaacttgtattcctcacttccttagctcctcctcttccttgttcctcctcttacaatggcaaaaatgcctttagagcctctaatagtggggagagtcataggagaagttcttgattctttcaccacaagcacaaaaatgactgtgagttacaacaagaagcaagtttacaatggccatgagctcttcccttccactgtcaacaccaagcccaaggttgagattgagggtggtgatatgaggtccttctttacactggtatatatatttcttttatctcttctcttcttattccttttcttctttaaagaacaagatttttttggggaaaaaaaaagagaaaaaacttaagctgttgccagtgtgtttctttgttattttctgtaatcatggtcacacggtatgtctcttttattggagttttctttacaaatactgaatcattaagcaaatgtccccctttttcggtgcagatcatgactgaccctgatgttcctggccctagtgacccttatctgagagagcacttgcactggtacttaatataaaaattaactaacttaagtagtcatttattacataaataacacaccacacccaccccacatatattgttataccggtttctcttaataacttaacctctttaaaagttatgtactggttatccatctgatattcacgttagatgtctgtgtattttattatcaagtttgaagttttgcaagatatatgcattcctttcatcacatcaaactatggacagccaggaaggtaataacaaatacttacatcacatgaagaaggtacacacatgcctttgtattctgcaaaagtagcttgttccaccataatcccatccaccctaaaatcaaaacaatctaaaagggtgaataaatctcgaagtctcaaacaaacattacagaactacatataaggcaaacaaccagtaaccattcattcattcataggtatctaacagtcaataattttgttgcaaacattctttttgctgcaggatagtgacagatattccaggcacaacagatgccacatttggtaggttcatatgaaaaatgtgtatatatgggaacttactttgatgatcatgagagagatcactgacaaaaaatgatattcctaataatatgtagggaaagagttggtgagctatgaggtcccaaagcctaatattggaatccataggtttgtgtttgtcctgttcaagcaaaagcgtagacagtgtgttactccacccacttcaagggaccacttcaacacacgcaaattcgcagcagagaacgaccttggcctccctgtggctgctgtctacttcaatgcacagagggaaacggctgcaagaagacgctagctatagctaagtagctgctggttttgccactgcaacaaccaaagtagtattgtattgaataaagcgataaaaaggtacaagtacaaggagtttcagtagtggaagttgatcttcacatgtggcttcaaataacttgcagaaagaaggaagataatcattttctagtttgactagtgtgtatgctatgttttactttccatcagatgttgtgtacgttctgtagtataagtgtactacgtgtattattattacctcgtgggtactactatgaggtgatcttatatatatatatataaagaaataagaggtttggtaggccatgtcgtttcttttcctttaatagtttagggtttggcttcgtttcttctttccttgcggtttcgatcactcagtaccttctctctattggtttttttcgatctcttcacaactaaattacgtttttgacagaaaaaatggtattattaattaattactatatgagactttatgatcagtgacttgtacacactttcgatcttgtcatgttgattatttttcctcaagatgcgaaacatttcactttcagtaatacgaggcttgctcgtctatataatatttatgacatctgctttttgctaacttgatggattgacccttcaggaaacagtgatttctacaccaaatttccagcatatattcacactgctaaaataacatgtgattagtgtgtatggaaactgttaatatgcactcctttaaatgcaggtgtaatcgttccaatgccctaaacatagaagctatgaaataaaatggcgttatgttgaaatcaacacatt

SEQ ID NO: 40 - DNA, 522 bp, Glycine max
  source 1..522: mol_type = other DNA
atggcaaaaatgcctttagagcctctaatagtggggagagtcataggagaagttcttgattctttcaccacaagcacaaaaatgactgtgagttacaacaagaagcaagtttacaatggccatgagctcttcccttccactgtcaacaccaagcccaaggttgagattgagggtggtgatatgaggtccttctttacactgatcatgactgaccctgatgttcctggccctagtgacccttatctgagagagcacttgcactggatagtgacagatattccaggcacaacagatgccacatttgggaaagagttggtgagctatgaggtcccaaagcctaatattggaatccataggtttgtgtttgtcctgttcaagcaaaagcgtagacagtgtgttactccacccacttcaagggaccacttcaacacacgcaaattcgcagcagagaacgaccttggcctccctgtggctgctgtctacttcaatgcacagagggaaacggctgcaagaagacgctag

SEQ ID NO: 41 - protein, 173 aa, Glycine max
MAKMPLEPLIVGRVIGEVLDSFTTSTKMTVSYNKKQVYNGHELFPSTVNTKPKVEIEGGDMRSFFTLIMTDPDVPGPSDPYLREHLHWIVTDIPGTTDATFGKELVSYEVPKPNIGIHRFVFVLFKQKRRQCVTPPTSRDHFNTRKFAAENDLGLPVAAVYFNAQRETAARRR

(19) United States
(12) Patent Application Publication
(10) Pub. No.: US 2025/0263452 A1
(43) Pub. Date: Aug. 21, 2025

(54) SOYBEAN JAG1 GENE MUTATIONS

(71) Applicant: INARI AGRICULTURE TECHNOLOGY, INC., Cambridge, MA (US)

(72) Inventors: Christine Marie Gault, Cambridge, MA (US); Rodrigo German Sala, Wildwood, MO (US)

(21) Appl. No.: 18/847,982

(22) PCT Filed: Mar. 20, 2023
(86) PCT No.: PCT/US2023/064702

§ 371 Date: Sep. 17, 2024

Related U.S. Application Data
(60) Provisional application No. 63/269,663, filed on Mar. 21, 2022.

(51) Int. Cl. C07K 14/415; C12N 9/22; C12N 15/11; C12Q 1/6806

(52) U.S. Cl. CPC C07K 14/415; C12N 9/226; C12N 15/11; C12Q 1/6806; C12N 2310/20

(57) ABSTRACT

The disclosure relates to novel plants, plant parts, and nucleotide sequences in soybean plants comprising a mutated JAG1 gene, along with methods of using and making the same. Wherein the mutated JAG1 gene comprises a null mutation in the JAG1 gene encoding the polypeptide of SEQ ID NO: 10 or an allelic variant thereof and wherein the soybean plant cell lacks a loss-of-function mutation in the soybean JAG2 gene.

### CROSS-REFERENCE TO RELATED APPLICATIONS

This international patent application claims the benefit of U.S. provisional patent application Ser. No. 63/269,663, filed Mar. 21, 2022.

### INCORPORATION OF SEQUENCE LISTING

The sequence listing contained in the file named P13530W000_ST26, which is 71,751 bytes measured in operating system Windows, created on Mar. 18, 2023, and electronically filed via Patent Center on Mar. 20, 2023, is incorporated herein by reference in its entirety.

### FIELD

Disclosed herein are novel plants, plant parts, and nucleotide sequences in soybean varieties comprising a mutated JAG1 gene, along with methods of making the same by growing a soybean plant or lot, and methods of using the same.

### BACKGROUND

Agriculture is an essential industry for the global economy and the United States in particular. Soybean (Glycine max) is an important legume crop worldwide due to its ability to fix atmospheric nitrogen. Soybeans serve as a major source of animal feed protein and soybean oil has uses in a wide variety of industries, including the food and beverage, biodiesel, and other industries.

Soybean sustainability is a priority for farmers worldwide. Farming practices such as water and nutrient management help farmers improve efficiencies, boost crop productivity, conserve water, enrich soil quality, improve nutrient efficiencies of the soil, and produce sustainable soybean crops. The benefits of bioengineering for soybean farmers include increased yields and extreme weather hardiness.

### SUMMARY

Disclosed herein are soybean plant cells comprising a mutated JAG1 gene, wherein the mutated JAG1 gene comprises a null mutation in the JAG1 gene encoding the polypeptide of SEQ ID NO: 10 or an allelic variant thereof and wherein the soybean plant cell lacks a loss-of-function mutation in the soybean JAG2 gene. Also disclosed herein are soybean plant cells comprising a mutated JAG1 gene, wherein the mutated JAG1 gene encodes the polypeptide comprising the amino acid sequence of SEQ ID NO: 12, 13, 14, 22, 24, 26, 28, 30, or an allelic variant thereof. In certain embodiments, the soybean plant cell is homozygous for the mutated JAG1 gene. In certain embodiments, the mutated JAG1 gene comprises the polynucleotide sequence of SEQ ID NO: 7, 8, 9, 15, 17, 19, 21, 23, 25, 27, 29, 31, 32, 33, 34, or 35 or an allelic variant thereof. In certain embodiments, the allelic variant of SEQ ID NO: 12, 13, 14, 22, 24, 26, 28, 30, is encoded by a JAG1 gene which comprises a deletion corresponding to nucleotides 426 to 430, 424 to 430, or 423 to 430 of the endogenous soybean JAG1 gene of SEQ ID NO: 1, respectively. Soybean plants, soybean plant parts, and soybean seed comprising any of the aforementioned soybean plant cells or seed as well as soybean seed lots comprising the soybean seed are provided. Methods of producing a soybean crop comprising planting a plurality of the aforementioned soybean seeds or an aforementioned seed lot are provided. Methods for producing a soybean by-product comprising at least one processing step of cleaning, cracking, flaking, crushing, macerating, pressing, extracting, expelling, and/or extruding an aforementioned seed lot are provided.

Polynucleotides comprising the sequence of SEQ ID NO: 7, 8, 9, 15, 17, 19, 21, 23, 25, 27, 29, 31, 32, 33, 34, or 35 or an allelic variant thereof or encoding the polypeptide comprising the amino acid sequence of SEQ ID NO: 12, 13, 14, 22, 24, 26, 28, 30, are provided. Biological samples comprising the polynucleotides are also provided.

Methods of producing a soybean seed lot comprising: (i) growing a population of soybean plants comprising a mutated JAG1 gene to maturity, wherein the mutated JAG1 gene encodes the polypeptide comprising the amino acid sequence of SEQ ID NO: 12, 13, 14, 22, 24, 26, 28, 30, or an allelic variant thereof and wherein the soybean plants are homozygous for the mutated JAG1 gene; and (ii) harvesting seed from the population of soybean plants of step (i) at maturity, thereby producing the soybean seed lot are provided.

Methods of making a soybean plant containing a mutated JAG1 gene comprising: (a) deleting: (i) nucleotides 426 to 430 of the endogenous soybean JAG1 gene of SEQ ID NO: 1 or an allelic variant thereof in a wild-type soybean plant cell; (ii) nucleotides 424 to 430 of the endogenous soybean JAG1 gene of SEQ ID NO: 1 or an allelic variant thereof in a wild-type soybean plant cell; or (iii) nucleotides 423 to 430 of the endogenous soybean JAG1 gene of SEQ ID NO: 1 or an allelic variant thereof in a wild-type soybean plant cell; to obtain a modified soybean plant cell comprising the mutated JAG1 gene; and (b) recovering a soybean plant from the modified soybean plant cell are provided.

### DESCRIPTIONS OF THE DRAWINGS

FIG. 1 shows the wild-type genomic DNA of the soybean JAG1 gene (SEQ ID NO: 1). Exons are in uppercase, introns are in lower case, and the translation initiation and termination codons are in uppercase, bold, italics, and underlined.

FIG. 2 shows a portion of the wild type JAG1 genomic DNA containing the end of the 2^{nd} exon and the beginning of the second intron of the soybean JAG1 gene. The annotated wild-type DNA sequence (SEQ ID NO: 5) indicates the exon and translated sequence fragment starting from Y17 (SEQ ID NO: 6), the intron region in lowercase, and the gRNA spacer complementary sequence with underlining. The deletions resulting in the frameshift mutations in the mJAG1 mutants of 1:5D (SEQ ID NO: 7), −2:7D (SEQ ID NO: 8), and −3:8D (SEQ ID NO: 9) are indicated by dashes in place of the deleted bases of the mutants.

### DETAILED DESCRIPTION

### Definitions

The phrase “allelic variant” as used herein refers to a polynucleotide or polypeptide sequence variant that occurs in a particular gene at particular locus in a different strain, variety, or isolate of a given organism.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the phrase “biological sample” refers to either intact or non-intact (e.g., milled soybean seed or soybean plant tissue, chopped soybean plant tissue, lyophilized tissue) soybean plant tissue. It may also be an extract comprising intact or non-intact seed or soybean plant tissue. The biological sample can comprise flour, meal, syrup, oil, starch, and cereals manufactured in whole or in part to contain soybean plant by-products. In certain embodiments, the biological sample is “non-regenerable” (i.e., incapable of being regenerated into a soybean plant or soybean plant part).

As used herein, the terms “correspond,” “corresponding,” and the like, when used in the context of an nucleotide position, mutation, and/or substitution in any given polynucleotide (e.g., an allelic variant of SEQ ID NO: 1) with respect to the reference polynucleotide sequence (e.g., SEQ ID NO: 1) all refer to the position of the polynucleotide residue in the given sequence that has identity to the residue in the reference nucleotide sequence when the given polynucleotide is aligned to the reference polynucleotide sequence using a pairwise alignment algorithm (e.g., CLUSTAL O 1.2.4 with default parameters).

As used herein, the terms “Cpf1” and “Cas12a” are used interchangeably to refer to the same RNA dependent DNA endonuclease (RdDe).

As used herein, the phrase “endogenous gene” refers to the native form of a gene in the genome of an organism.

As used herein, the terms “include,” “includes,” and “including” are to be construed as at least having the features to which they refer while not excluding any additional unspecified features. As used herein, the term “plant” includes reference to an immature or mature whole soybean plant, including a plant from which seed or grain or anthers have been removed. Any seed or embryo that will produce the plant is also considered to be the soybean plant.

As used herein, the phrase “loss-of-function” mutation or allele of a gene refers to a mutation or allele of a gene which exhibits decreased gene function in comparison to an unmutated or wild-type allele of the gene. “Loss-of-function” mutations thus include mutations resulting in both null (i.e., amorphic) alleles and hypomorphic alleles (i.e., reduced by not eliminated function) of a gene.

As used herein, the phrase “mutated JAG1 gene” or “mJAGT gene” refer to an endogenous soybean JAG1 gene comprising a null mutation. In certain embodiments, the mJAGT comprising the null mutation encodes the polypeptide of SEQ ID NO: 12, 13, 14, 22, 24, 26, 28, 30, or allelic variants thereof, wherein the allelic variants of SEQ ID NO: 12, 13, 14, 22, 24, 26, 28, or 30 comprise deletions corresponding to deletions of the amino acid residues encoded by nucleotides 426 to 430, 424 to 430, or 423 to 430 of the endogenous soybean JAG1 gene of SEQ ID NO: 1, respectively. In certain embodiments, the mJAGT deletion is a null mutation that comprises a deletion corresponding to at least nucleotides 421 to 436 of SEQ ID NO: 1.

As used herein, a null mutation of a gene refers to an allele of a gene having no gene activity in comparison to the wild-type allele of the gene. Null alleles are also known as amorphic alleles.

As used herein, the term “plant” includes a whole soybean plant and any descendant, cell, tissue, part, or parts of the plant. The term “plant” thus includes reference to an immature or mature whole soybean plant, including a plant from which seed or grain or anthers have been removed.

The term “plant parts” include any part(s) of a plant, including, for example and without limitation: seed (including mature seed and immature seed); grain; stover; a plant cutting; a plant cell; a plant cell culture; or a plant organ (e.g., pollen, embryos, pods; flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, flowers, or stalks. In contrast, some plant cells are not capable of being regenerated to produce plants and are referred to herein as “non-regenerable” plant cells.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

The present disclosure provides for the soybean plant cells, plant parts including seed, plants, and biological samples comprising a mJAG1 gene. These mJAGiplants and parts can be utilized for human food, livestock feed, as a raw material in industry, or as breeding material for development of other soybean varieties. The target endogenous JAG1 gene comprises the genomic DNA of SEQ ID NO: 1 and allelic variants thereof located on soybean chromosome 20. The endogenous soybean JAG1 gene is located at nucleotides 35827671 to 35830107 of chromosome 20 of the Glycine max Wm82.a2.v1 set forth in the https internet site “phytozome-next.jgi.doe.gov/report/transcript/Gmax_Wm82_a2_v1/Glyma.20G116200.1.” Allelic variants of an endogenous soybean JAG1 gene include variants which encode JAG1 proteins having at least 95%, 96%, 98%, 99%, or 99.5% sequence identity to SEQ ID NO: 10. Allelic variants of an endogenous soybean JAG1 gene also include variants which comprise genomic DNA having at least 95%, 96%, 98%, 99%, or 99.5% sequence identity to SEQ ID NO: 1. In certain embodiments, mJAG1 genes can encode the polypeptide comprising the amino acid sequence of SEQ ID NO: 12, 13, 14, 22, 24, 26, 28, 30, or an allelic variant thereof. In certain embodiments, such allelic variants of SEQ ID NO: 12, 13, or 14 can comprise an amino acid sequence having at least 95%, 96%, 98%, 99%, or 99.5% sequence identity across the entire length of SEQ ID NO: 12, 13, 14, 22, 24, 26, 28, and 30. In certain embodiments, such allelic variants of SEQ ID NO: 12, 13, 14, 22, 24, 26, 28, or 30 can comprise an amino acid sequence having at least 95%, 96%, 98%, 99%, or 99.5% sequence identity across the entire length of SEQ ID NO: 12, 13, and 14 and are encoded by a JAG1 gene which comprises a deletion corresponding to nucleotides 426 to 430, 424 to 430, or 423 to 430 of the endogenous soybean JAG1 gene of SEQ ID NO: 1, respectively. In certain embodiments, the mJAG1 gene can comprise a JAG1 gene or an aforementioned allelic variant thereof which comprises a deletion corresponding to nucleotides 426 to 430, 424 to 430, or 423 to 430 of the endogenous soybean JAG1 gene of SEQ ID NO: 1. In certain embodiments, the mJAG1 deletion is a null mutation that comprises a deletion corresponding to at least nucleotides 421 to 436 of SEQ ID NO: 1 or an allelic variant thereof.

In certain embodiments, the soybean plant cells, plants, plant parts, and seeds comprising mJAG1 null alleles of the JAG1 gene lack a loss-of-function mutation (e.g., a null mutation) in the soybean JAG2 gene. The wild-type endogenous soybean JAG2 gene, as referred to as GmJAG2, comprises the genomic DNA of SEQ ID NO: 36 or an allelic variant thereof located on soybean chromosome 10. The endogenous soybean JAG2 gene is located at nucleotides 49647129 to 49649853 of chromosome 10 of the Glycine max Wm82.a2.v1 genome set forth in the https internet site “soybase.org” under the gene identifier Glyma.10g273800 and comprises the coding sequence of SEQ ID NO: 37 which encodes the wild-type JAG2 protein of SEQ ID NO: 38. Soybean plant cells, plants, plant parts, and seeds lacking a loss-of-function mutation (e.g., a null mutation) in the soybean JAG2 gene can at least be identified by sequencing their genomic DNA regions correspond to SEQ ID NO:36.

In certain embodiments, the mJAG1 null alleles of the JAG1 gene provided herein will provide for soybean plants which exhibit both a narrow leaf phenotype and an increased number of seeds per kilogram of seeds harvested from the soybean plant homozygous for the null allele of the mJAG1 gene, where both the narrow leaf phenotype and increased number of seeds per kilogram of seeds harvested are in comparison to the leaf phenotype and the number of seeds per kilogram harvested from corresponding control soybean plant lacking the mJAG1 gene (e.g., a wild-type soybean plant homozygous for a wild-type JAG1 gene). In certain embodiments, the soybean plants homozygous for the mJAG1 null alleles will lack a loss-of-function mutation in the soybean JAG2 gene and will exhibit the aforementioned narrow leaf and an increased number of seeds per kilogram of seeds phenotypes in comparison to the control plant.

In certain embodiments, soybean plants homozygous for an mJAG1 gene can yield seed lots wherein the number of seeds per kilogram of seeds harvested from the soybean plant homozygous for the mJAG1 gene is increased in comparison to the number of seeds per kilogram harvested from corresponding control soybean plant lacking the mJAG1 gene (e.g., a wild-type soybean plant homozygous for a wild-type JAG1 gene). In certain embodiments, the number of seeds per kilogram of seeds harvested from the soybean plant homozygous for the mJAG1 gene is increased by up to about 5%, 6%, 7%, 8%, 9%, 10%, or 12% in comparison to the number of seeds per kilogram harvested from corresponding control soybean plant lacking the mJAG1 gene (e.g., a wild-type soybean plant homozygous for a wild-type JAG1 gene). In certain embodiments, the number of seeds per kilogram of seeds harvested from the soybean plant homozygous for the mJAG1 gene is increased by about 1% or 2% to any one of about 5%, 6%, 7%, 8%, 9%, 10%, or 12% in comparison to the number of seeds per kilogram harvested from corresponding control soybean plant lacking the mJAG1 gene (e.g., a wild-type soybean plant homozygous for a wild-type JAG1 gene). In certain embodiments, the weight in grams per 1000 seeds harvested from the soybean plant homozygous for the mJAG1 gene is decreased by up to about 2%, 4%, 6%, 8%, 9%, 10%, or 11% in comparison to the weight in grams per 1000 seeds harvested from corresponding control soybean plant lacking the mJAG1 gene (e.g., a wild-type soybean plant homozygous for a wild-type JAG1 gene). In certain embodiments, the weight in grams per 1000 seeds harvested from the soybean plant homozygous for the mJAG1 gene is decreased by up to about 2%, 4%, 6%, 8%, 9%, 10%, or 11% in comparison to the weight in grams per 1000 seeds harvested from corresponding control soybean plant lacking the mJAG1 gene (e.g., a wild-type soybean plant homozygous for a wild-type JAG1 gene). In certain embodiments of any of the aforementioned seed lots, the mJAG1 gene encodes the polypeptide comprising the amino acid sequence of SEQ ID NO: 12 or an allelic variant thereof. In certain embodiments, such allelic variants of SEQ ID NO: 12 can comprise an amino acid sequence having at least 95%, 96%, 98%, 99%, or 99.5% sequence identity across the entire length of SEQ ID NO: 12. In certain embodiments, such allelic variants of SEQ ID NO: 12 can comprise an amino acid sequence having at least 95%, 96%, 98%, 99%, or 99.5% sequence identity across the entire length of SEQ ID NO: 12 and are encoded by an mJAG1 gene which comprises a deletion corresponding to nucleotides 426 to 430 of the endogenous soybean JAG1 gene of SEQ ID NO: 1. In certain embodiments, the mJAG1 gene can comprise an mJAG1 gene or an aforementioned allelic variant thereof which comprises a deletion corresponding to nucleotides 426 to 430 of the endogenous soybean JAG1 gene of SEQ ID NO: 1.

Also provided here are polynucleotides comprising any of the aforementioned mJAG1 genes or fragments thereof. In certain embodiments, a polynucleotide comprising the sequence of SEQ ID NO: 7, 8, 9, 15, 16, 17, 18, 19, 21, 23, 25, 27, 29, 31, 32, 33, 34, 35, or allelic variants thereof are provided. In certain embodiments, the allelic variants of SEQ ID NO: 7, 8, or 9 will comprise sequences having at least 95%, 97%, 98%, 99%, or 99.5% sequence identity across the entire length of SEQ ID NO: 7, 8, 9, 15, 16, 17, 18, or 19 with the proviso that the sequences are not identical to across their entire length to SEQ ID NO: 1 and comprise a deletion corresponding to nucleotides 426 to 430, 424 to 430, or 423 to 430 of the endogenous soybean JAG1 gene of SEQ ID NO: 1. In certain embodiments, the allelic variants of SEQ ID NO: 21, 23, 25, 27, 29, 31, 32, 33, 34, or 35 will comprise sequences having at least 95%, 97%, 98%, 99%, or 99.5% sequence identity across the entire length of SEQ ID NO: 21, 23, 25, 27, 29, 31, 32, 33, 34, 35 with the proviso that the sequences are not identical to across their entire length to SEQ ID NO: 1 and comprise deletions in the SEQ ID NO: 1 sequence corresponding to the deletions of SEQ ID NO: 21, 23, 25, 27, or 29. In certain embodiments, the polynucleotides encode a polypeptide comprising the amino acid sequence of SEQ ID NO: 12, 13, 14, 22, 24, 26, 28, 30, or an allelic variant thereof. In certain embodiments, the encoded allelic variant will comprise a polypeptide having at least at least 95%, 97%, 98%, 99%, or 99.5% sequence identity across the entire length of SEQ ID NO: 12, 13, 14, 22, 24, 26, 28, or 30 with the proviso that the sequences are not identical to SEQ ID NO: 10. In certain embodiments, the polynucleotide is an isolated polynucleotide. Biological samples and soybean by-products comprising any of the aforementioned polynucleotides are also provided. In certain embodiments, the by-products are processed products are made from the mJAG1 soybean plant or its seeds, including: (a) soybean seed meal (defatted or non-defatted); (b) extracted soybean proteins, oils, sugars, syrups, and starches; (c) soy fermentation products; (d) soybean based animal feed or human food products (e.g., feed and food comprising soybean seed meal (defatted or non-defatted) and other ingredients (e.g., other cereal grains, other seed meal, other protein meal, other oil, other starch, other sugar, a binder, a preservative, a humectant, a vitamin, and/or mineral); (e) a pharmaceutical; (f) raw or processed biomass (e.g., cellulosic and/or lignocellulosic material; silage); and (g) various industrial products.

Methods of using the mJAG1 soybean plants, seeds, and seed lots to produce soybean by-products are also provided. Such methods will typically include at least one processing step of cleaning, cracking, flaking, crushing, macerating, pressing, extracting, expelling, and/or extruding the seed.

This disclosure also is directed to methods for producing a soybean plant having a mutated JAG1 gene by crossing a first parent soybean plant with a second parent soybean plant wherein the first or second parent soybean plant is a mJAG1 plant. Further, both first and second parent soybean plants can come from the mJAG1 plant. Still further, this disclosure also is directed to methods for producing mJAG1 plant-derived soybean plant by crossing a mJAG1 plant with a soybean plant, growing the progeny seed, and repeating the crossing and growing steps with the mJAG1 plant-derived soybean plant from 1 to 2 times, 1 to 3 times, 1 to 4 times, or 1 to 5 times. Thus, any such methods using a mJAG1 plant are part of this disclosure: selfing, backcrosses, hybrid production, crosses to populations, and the like. All plants produced using a mJAG1 plant as a parent are within the scope of this disclosure, including plants derived from a mJAG1 plant having a mutated JAG1 gene. Also provided are the F1 progeny soybean plant produced from the crossing of a mJAG1 containing soybean plant (e.g., a plant homozygous for the mJAG1 gene) with any other soybean plant, F1 seed, and various parts of the F1 soybean plant.

The following describes breeding methods that may be used with a mJAG1 plant in the development of further soybean plants. One such embodiment is a method for developing a mJAG1 progeny soybean plant in a soybean plant breeding program comprising: obtaining the soybean plant, or its parts, of a mJAG1 plant and utilizing said plant or plant parts as a source of breeding material; and selecting a mJAG1 progeny plant having the mutated JAG1 (mJAG1) gene. Breeding steps that may be used in the soybean plant breeding program include pedigree breeding, backcrossing, mutation breeding, and recurrent selection. In conjunction with these steps, techniques such as restriction fragment polymorphism enhanced selection, genetic marker enhanced selection (for example SSR markers), and the making of double haploids may be utilized.

Another method involves producing a population of mJAG1 progeny soybean plants, comprising crossing a mJAG1 plant with another soybean plant, thereby producing a population of soybean plants, which, on average, derive 50% of their alleles from the mJAG1 plant. A mJAG1 plant of this population may be selected and repeatedly selfed or sibbed, with a soybean variety resulting from these successive filial generations. One embodiment of this disclosure is the soybean variety produced by this method and that has obtained at least 50% of its alleles from mJAG1.

Field crops are bred through techniques that take advantage of the plant's method of pollination. According to the disclosure, a mJAG1 plant may be crossed with self-pollinated, sib-pollinated, or cross pollinated to create a pedigree soybean plant. A plant is self-pollinated if pollen from one flower is transferred to the same or another flower of the same plant. A plant is sib-pollinated when individuals within the same family or variety are used for pollination. A plant is cross-pollinated if the pollen comes from a flower on a different plant from a different family or variety. The terms “cross-pollination” and “out-cross” as used herein do not include self-pollination or sib-pollination. Soybean plants (Glycine max) are recognized to be naturally self-pollinated plants which, while capable of undergoing cross-pollination, rarely do so in nature. Insects are reported by some researchers to carry pollen from one soybean plant to another and it generally is estimated that less than one percent of soybean seed formed in an open planting can be traced to cross-pollination, i.e. less than one percent of soybean seed formed in an open planting is capable of producing F1 hybrid soybean plants,

Any other suitable breeding, selection, or growing methods may be used. Choice of the particular breeding or selection method with vary depending on environmental factors, population size, and the like.

In certain embodiments, soybean plant cells, plant parts (e.g., seeds), and plants comprising a mJAG1 gene and a transgenic locus are provided. In one embodiment, an mBS1 or mBS1 gene is combined with one or more soybean GM events providing tolerance to any one or a combination of glyphosate-based, glufosinate-based, HPPD inhibitor-based, sulfonylurea- or imidazolinone-based, AHAS- or ALS-inhibiting and/or auxin-type (e.g., dicamba, 2,4-D) herbicides and/or an insect resistance trait, such as Event EE-GM3 (aka FG-072, MST-FGØ72-3, described in WO2011063411, USDA-APHIS Petition 09-328-01p), Event SYHTOH2 (aka 0H2, SYN-ØØØH2-5, described in WO2012/082548 and 12-215-01p), Event DAS-68416-4 (aka Enlist Soybean, described in WO2011/066384 and WO2011/066360, USDA-APHIS Petition 09-349-01p), Event DAS-44406-6 (aka Enlist E3, DAS-444Ø6-6, described in WO2012/075426 and USDA-APHIS 11-234-01p), Event MON87708 (dicamba-tolerant event of Roundup Ready 2 Xtend Soybeans, described in WO2011/034704 and USDA-APHIS Petition 10-188-01p, MON-877Ø8-9), Event MON89788 (aka Genuity Roundup Ready 2 Yield, described in WO2006/130436 and USDA-APHIS Petition 06-178-01p), Event 40-3-2 (aka Roundup Ready, GTS 40-3-2, MON-Ø4Ø32-6, described in USDA-APHIS Petition 93-258-01), Event A2704-12 (aka LL27, ACS-GMØØ5-3, described in WO2006108674 and USDA-APHIS Petition 96-068-01p), Event 127 (aka BPS-CV127-9, described in WO2010/080829), Event A5547-127 (aka LL55, ACS-GMØØ6-4, described in WO2006108675 and in USDA-APHIS Petition 96-068-01p), event MON877Ø5 (MON-87705-6, Vistive Gold, published PCT patent application WO2010/037016, USDA-APHIS Petition 09-201-01p), or event DP3Ø5423 (aka DP-3Ø5423-1, published PCT patent application WO2008/054747, USDA-APHIS Petition 06-354-01p), or EE-GM5 is combined with a combination of the following events: Event MON98788×MON87708 (aka Roundup Ready 2 Xtend Soybeans, MON-877Ø8-9×MON-89788-1), Event HOS×Event 40-3-2 (aka Plenish High Oleic SoybeansxRoundup Ready Soybeans), Event EE-GM3×EE-GM2 (aka FG-072xLL55, described in WO2011063413), Event MON 87701×MON 89788 (aka Intacta RR2 Pro Soybean, MON-877Ø1-2×MON-89788-1), DAS-81419-2×DAS-44406-6 (aka Conkesta™ Enlist E3™ Soybean, DAS-81419-2×DAS-444Ø6-6), Event DAS-68416-4×Event MON 89788 (aka Enlist™ RoundUp Ready® 2 Soybean, DAS-68416-4×MON-89788-1), Event MON-87769-7×Event MON-89788-1 (aka Omega-3×Genuity Roundup Ready 2 Yield Soybeans), Event MON 87705×Event MON 89788 (aka Vistive Gold, MON-877Ø5-6×MON-89788-1), or Event MON87769×Event MON89788 (aka Omega-3×Genuity Roundup Ready 2 Yield Soybeans, MON-87769-7×MON-89788-1), where all published PCT patent applications or US national stages thereof are incorporated herein by reference in there entireties. Representative transgenic events that can be combined with an mJAG1 gene include those set forth in Table 1. Also provided herein are soybean plant cells, plant parts (e.g., seeds), and plants comprising an mJAG1 gene and a modification of any of the aforementioned transgenic events or transgenic events set forth in Table 1 below. Modifications of the transgenic events include those disclosed in WO2022/026375, WO2022/026379, WO2022/026390, WO2022/026395, WO2022/026403, US Patent Applic. Pub. No. US20220030822, and U.S. Pat. No. 11,242,534, which are each incorporated herein by reference in their entireties.

TABLE 1
Transgenic Soybean Events

| | | ATCC;³ NCIMB⁴ | |
|---|---|---|---|
| | Patent or Patent | Deposit Number; | Trait |
| Event Name | Application | or Commercial | expression |
| (traits)¹ | Number(s)² | Source | cassette(s) |
| A5547-127 (HT) | US 20080196127 | NCIMB 41660 | PAT |
| | RE44962 | | |
| DAS44406-6 | U.S. Pat. No. 9,540,655 | PTA-11336 | Aad-12, |
| (HT)⁵ | U.S. Pat. No. 10,400,250 | | 2mepsps, PAT |
| DAS68416-4 | U.S. Pat. No. 9,738,904 | PTA-10442 | Aad-12, PAT |
| (IR, HT)⁶ | | PTA-12006 | |
| DAS81419-2 | U.S. Pat. No. 8,680,363 | PTA-12006 | cry1Ac, cry1F, |
| (IR, HT) | U.S. Pat. No. 8,632,978 | | PAT |
| | U.S. Pat. No. 9,695,441 | | |
| | U.S. Pat. No. 9,738,904 | | |
| GTS 40-3-2 | US 20070136836 | M690GT \| 0.9 RM | cp4epsps |
| (HT) | | Soybean⁷ | |
| MON87701 (IR) | U.S. Pat. No. 8,049,071 | PTA-8194 | cry1Ac |
| MON87708 | U.S. Pat. No. 9,447,428 | PTA-9670 | DMO |
| (HT)⁸ | | | |
| MON89788 | U.S. Pat. No. 9,944,945 | PTA-6708 | cp4epsps |
| (HT) | | | |
| MST-FGØ72-3 | U.S. Pat. No. 8,592,650 | NCIMB 41659 | hppdPF W336, |
| (HT)⁹ | | | 2mepsps |
| SYHT0H2¹⁰ | U.S. Pat. No. 10,184,134 | PTA-11226 | cAvHPPD-03 |

¹Traits: IR = Insect Resistance; HT = Herbicide Tolerance; AR = Antibiotic Resistance;
²Each US Patent or Patent Application Publication is incorporated herein by reference in its entirety.
³ATCC is the American Type Culture Collection, 10801 University Boulevard Manassas, VA 20110 USA (for “PTA-XXXXX” deposits).
⁴NCIMB is the National Collection of Industrial, Food and Marine Bacteria, Ferguson Building, Craibstone Estate, Bucksburn, Aberdeen AB9YA, Scotland.
⁵HT to 2,4-D; glyphosate, and glufosinate; also refered to as pDAB8264.44.06.1.
⁶Independent IR/HT and HT events combined by breeding. IR/HT event (Cry1F, Cry1Ac synpro (Cry1Ac), and PAT) is DAS81419-2, deposited with ATCC under PTA-12006, also referred to as DAS81419-2.
⁷Elk Mound Seed, 308 Railroad Street Elk Mound, WI, USA 54739.
⁸HT to dicamba.
⁹HT to both glyphosate and isoxaflutole herbicides.
¹⁰HT to glufosinate and mesotrione herbicides.

In certain embodiments, a mutated Jag1 gene is combined with a null mutation in the soybean TFL1b gene which is an ortholog of the Arabidopsis Terminal Flower 1 gene. The soybean TFL1b ortholog is found in the https internet database “soybase.org” as Glyma.19G194300 and also known as the soybean stem growth habit gene Dtl (Liu et al. Plant Physiol. 2010 May; 153(1):198-210. doi: 10.1104/pp. 109.150607. Epub 2010 Mar. 10. PMID: 20219831; PMCID: PMC2862436). The TFL1b wild-type genomic DNA is provided as SEQ ID NO: 39, the TFL1b wild-type coding sequence is provided as SEQ ID NO: 40, and the TFL1b protein is provided as SEQ ID NO: 41. Null mutations in TFL1b can be obtained by gene editing techniques (e.g., by use of CRISPR/Cas9, CRISPR/Cas12, TALEN, or aZFN-mediated site-specific mutagenesis of the TFL1b gene).

Methods of producing a soybean seed lot comprising: (i) growing a population of soybean plants comprising a mJAG1 gene to maturity; and (ii) harvesting seed from the population of soybean plants of step (i) at maturity, thereby producing the soybean seed lot, wherein the soybean plants are homozygous for the mutated JAG1 gene. In certain embodiments, the seed lot is packaged in lots comprising about 50 to 60 pounds (e.g. about 22.7 to 27.2 kilograms). In certain embodiments, the seed lots comprised of seed homozygous for certain mJAG1 mutations will contain more seed than control seed lots obtained from control soybeans which comprise a wild-type JAG1 gene. In certain embodiments, up to about 2%, 4%, 5%, 6%, 7%, 8%, 9%, 10%, 11%, or 12% more seed will be present in the seed lots comprised of seed homozygous for certain mJAG1 mutations in comparison to control seed lots obtained from control soybeans which comprise a wild-type JAG1 gene. Also provided herein are methods of treating the mJAG1 seeds and seed lots and the resultant treated mJAG1 seed and seed lots. Seeds can be treated with such fertilizers, biological agents, nematicides, insecticides, and fungicides by methods including in-furrow applications or by coating (e.g., with a drum coater, rotary coater, tumbling drum, fluidized bed, and/or spouted bed apparatus). Methods and compositions including various binders, fillers, film coats, and active ingredients such as fertilizers, surfactants, plant growth regulators, crop desiccants, fungicides, bacteriocides, bacteriostats, insecticides, and insect repellants for coating seeds that can be adapted for use with seeds provided herein are disclosed in U.S. patent Ser. No. 10/745,578, which is incorporated herein by reference in its entirety.

The disclosure also provides a method of making a soybean plant containing a mutated JAG1 gene. In certain embodiments, the methods can comprise making deletions or combinations of a deletions with insertions and/or substitutions which result in an mJAG1 gene encoding a polypeptide of SEQ ID NO: 12, 13, 14, 22, 24, 26, 28, 30, or an allelic variant thereof. Gene editing molecules of use in methods provided herein include molecules capable of introducing a double-strand break (“DSB”) or single-strand break (“SSB”) at a specific site or sequence in a double-stranded DNA, such as in genomic DNA or in a target gene located within the genomic DNA as well as accompanying guide RNA. In certain embodiments, the mJAG1 allele results from introduction of a DSB at a target site in the JAG1 gene (e.g., SEQ ID NO: 1 or an allelic variant thereof) to induce non-homologous end joining (NHEJ) at the site of the break followed by recovery of desired mJAG1 alleles. In certain embodiments, the mJAG1 allele results from introduction of a DSB at a target site in the JAG1 gene (e.g., SEQ ID NO: 1 or an allelic variant thereof) followed by homology-directed repair (HDR), microhomology-mediated end joining (MMEJ), or NHEJ to introduce a desired donor or other DNA template polynucleotide at the DSB, followed by recovery of the desired mJAG1 allele. Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a type II Cas nuclease, a Cas9, a nCas9 nickase, a type V Cas nuclease, a Cas12a nuclease, a nCas12a nickase, a Cas12d (CasY), a Cas12e (CasX), a Cas12b (C2c1), a Cas12c (C2c3), a Cas12i, a Cas12j, a Cas14, an engineered nuclease, a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for use with an RNA-guided nuclease, or a DNA encoding a gRNA for use with an RNA-guided nuclease; (d) optionally donor DNA template polynucleotides suitable for insertion at a break in genomic DNA by homology-directed repair (HDR) or microhomology-mediated end joining (MMEJ); and (e) optionally other DNA templates (e.g., dsDNA, ssDNA, or combinations thereof) suitable for insertion at a break in genomic DNA (e.g., by non-homologous end joining (NHEJ). In certain embodiments, the methods can comprising: (a) deleting: (i) nucleotides 426 to 430 of the endogenous soybean JAG1 gene of SEQ ID NO: 1 or an allelic variant thereof in a wild-type soybean plant cell; (ii) nucleotides 424 to 430 of the endogenous soybean JAG1 gene of SEQ ID NO: 1 or an allelic variant thereof in a wild-type soybean plant cell; or (iii) nucleotides 423 to 430 of the endogenous soybean JAG1 gene of SEQ ID NO: 1 or an allelic variant thereof in a wild-type soybean plant cell; to obtain a modified soybean plant cell comprising the mutated JAG1 gene; and (b) recovering a soybean plant from the modified soybean plant cell.

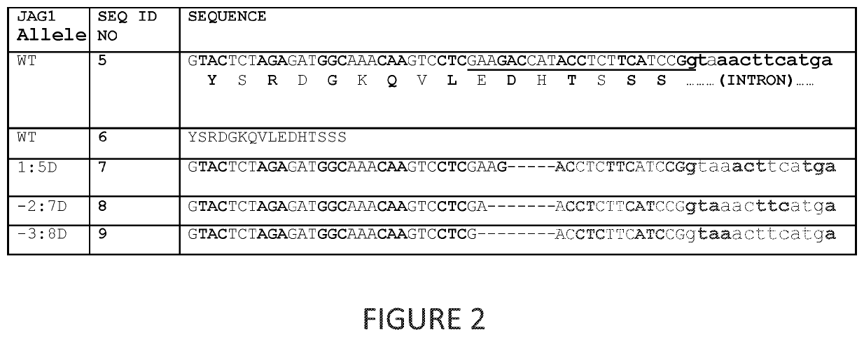

In certain embodiments, the mJAG1 gene and plant cells, parts including seeds, and plants comprising the mJAG1 gene are generated by CRISPR technology. CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application 61/945,700). Methods of using CRISPR technology for genome editing in plants are disclosed in US Patent Application Publications US 2015/0082478A1 and US 2015/0059010A1 and in International Patent Application PCT/US2015/038767 A1 (published as WO 2016/007347 and claiming priority to U.S. Provisional Patent Application 62/023,246). In certain embodiments, an RNA-guided endonuclease that leaves a blunt end following cleavage of the target site is used. Blunt-end cutting RNA-guided endonucleases include Cas9, Cas12c, Cas12i, and Cas12h (Yan et al., 2019). In certain embodiments, an RNA-guided endonuclease that leaves a staggered single stranded DNA overhanging end following cleavage of the target site following cleavage of the target site is used. Staggered-end cutting RNA-guided endonucleases include Cas12a, Cas12b, and Cas12e. A non-limiting target Cas12 cleavage site region in the JAG1 gene set forth in SEQ ID NO: 1 and SEQ ID NO: 5 is noted in FIG. 2. Guide RNAs comprising a spacer RNA encoded by SEQ ID NO: 2 can be used in conjunction with Cas12 nucleases to generate mJAG1 genes which: (i) encode the polypeptides of SEQ ID NO: 12, 13, 14, 22, 24, 26, 28, 30, or an allelic variant thereof or an allelic variant thereof; (ii) comprise a deletion in an endogenous JAG1 gene of SEQ ID NO: 1 or an allelic variant thereof corresponding a deletion of SEQ ID NO: 7, 8, 9, 21, 23, 25, 27, 29, 31, 32, 33, 34, 35, or an allelic variant thereof; and/or (ii) comprise deletions of nucleotides 426 to 430 of the endogenous soybean JAG1 gene of SEQ ID NO: 1 or an allelic variant thereof, deletions of nucleotides 424 to 430 of the endogenous soybean JAG1 gene of SEQ ID NO: 1 or an allelic variant thereof in a wild-type soybean plant cell, or deletions of nucleotides 423 to 430 of the endogenous soybean JAG1 gene of SEQ ID NO: 1 or an allelic variant thereof. All of the patent publications referenced in this paragraph are incorporated herein by reference in their entirety.

CRISPR-type genome editing can be adapted for use in the plant cells and methods provided herein in several ways. CRISPR elements, e.g., gene editing molecules comprising CRISPR endonucleases and CRISPR guide RNAs including single guide RNAs or guide RNAs in combination with tracrRNAs or scoutRNA, or polynucleotides encoding the same, are useful in effectuating genome editing without remnants of the CRISPR elements or selective genetic markers occurring in progeny. In certain embodiments, the CRISPR elements are provided directly to the eukaryotic cell (e.g., soybean plant cells), systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, soybean plants or soybean plant cells used in the systems, methods, and compositions provided herein can comprise a transgene that expresses a CRISPR endonuclease (e.g., a Cas9, a Cpf1-type or other CRISPR endonuclease). In certain embodiments, one or more CRISPR endonucleases with unique PAM recognition sites can be used. Guide RNAs (sgRNAs or crRNAs and a tracrRNA) to form an RNA-guided endonuclease/guide RNA complex which can specifically bind sequences in the gDNA target site that are adjacent to a protospacer adjacent motif (PAM) sequence. The type of RNA-guided endonuclease typically informs the location of suitable PAM sites and design of crRNAs or sgRNAs. G-rich PAM sites, e.g., 5′-NGG are typically targeted for design of crRNAs or sgRNAs used with Cas9 proteins. Examples of PAM sequences include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). T-rich PAM sites (e.g., 5′-TTN or 5′-TTTV, where “V” is A, C, or G) are typically targeted for design of crRNAs or sgRNAs used with Cas12 proteins. In some instances, a Cas12 nuclease such as Cas12a can also recognize a 5′-CTA PAM motif. Other examples of potential Cas12 PAM sequences include TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Cpf1 endonuclease and corresponding guide RNAs and PAM sites are disclosed in US Patent Application Publication 2016/0208243 A1, which is incorporated herein by reference for its disclosure of DNA encoding Cpf1 endonucleases and guide RNAs and PAM sites.

In certain embodiments, the mJAG1 gene and plant cells, parts including seeds, and plants comprising the mJAG1 gene are generated by use of zinc finger nucleases or zinc finger nickases. Zinc-finger nucleases are site-specific endonucleases comprising two protein domains: a DNA-binding domain, comprising a plurality of individual zinc finger repeats that each recognize between 9 and 18 base pairs, and a DNA-cleavage domain that comprises a nuclease domain (typically Fok1). The cleavage domain dimerizes in order to cleave DNA; therefore, a pair of ZFNs are required to target non-palindromic target polynucleotides. In certain embodiments, zinc finger nuclease and zinc finger nickase design methods which have been described (Umov et al. (2010) Nature Rev. Genet., 11:636-646; Mohanta et al. (2017) Genes vol. 8, 12: 399; Ramirez et al. Nucleic Acids Res. (2012); 40(12): 5560-5568; Liu et al. (2013) Nature Communications, 4: 2565) can be adapted for use in the methods set forth herein. The zinc finger binding domains of the zinc finger nuclease or nickase provide specificity and can be engineered to specifically recognize any desired target DNA sequence. The zinc finger DNA binding domains are derived from the DNA-binding domain of a large class of eukaryotic transcription factors called zinc finger proteins (ZFPs). The DNA-binding domain of ZFPs typically contains a tandem array of at least three zinc “fingers” each recognizing a specific triplet of DNA. A number of strategies can be used to design the binding specificity of the zinc finger binding domain. One approach, termed “modular assembly”, relies on the functional autonomy of individual zinc fingers with DNA. In this approach, a given sequence is targeted by identifying zinc fingers for each component triplet in the sequence and linking them into a multifinger peptide. Several alternative strategies for designing zinc finger DNA binding domains have also been developed. These methods are designed to accommodate the ability of zinc fingers to contact neighboring fingers as well as nucleotide bases outside their target triplet. Typically, the engineered zinc finger DNA binding domain has a novel binding specificity, compared to a naturally occurring zinc finger protein. Engineering methods include, for example, rational design and various types of selection. Rational design includes, for example, the use of databases of triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, e.g., U.S. Pat. Nos. 6,453,242 and 6,534,261, both incorporated herein by reference in their entirety. Exemplary selection methods (e.g., phage display and yeast two-hybrid systems) can be adapted for use in the methods described herein. In addition, enhancement of binding specificity for zinc finger binding domains has been described in U.S. Pat. No. 6,794,136, incorporated herein by reference in its entirety. In addition, individual zinc finger domains may be linked together using any suitable linker sequences. Examples of linker sequences are publicly known, e.g., see U.S. Pat. Nos. 6,479,626; 6,903,185; and 7,153,949, incorporated herein by reference in their entirety. The nucleic acid cleavage domain is non-specific and is typically a restriction endonuclease, such as Fok1. This endonuclease must dimerize to cleave DNA. Thus, cleavage by Fok1 as part of a ZFN requires two adjacent and independent binding events, which must occur in both the correct orientation and with appropriate spacing to permit dimer formation. The requirement for two DNA binding events enables more specific targeting of long and potentially unique recognition sites. Fok1 variants with enhanced activities have been described and can be adapted for use in the methods described herein; see, e.g., Guo et al. (2010) J. Mol. Biol., 400:96-107.

In certain embodiments, the mJAG1 gene and plant cells, parts including seeds, and plants comprising the mJAG1 gene are generated by use of TAL-effector nucleases or TALENs. Transcription activator like effectors (TALEs) are proteins secreted by certain Xanthomonas species to modulate gene expression in host plants and to facilitate the colonization by and survival of the bacterium. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site has been found. The simple relationship between amino acid sequence and DNA recognition of the TALE binding domain allows for the design of DNA binding domains of any desired specificity. TALEs can be linked to a non-specific DNA cleavage domain to prepare genome editing proteins, referred to as TAL-effector nucleases or TALENs. As in the case of ZFNs, a restriction endonuclease, such as Fok1, can be conveniently used. Methods for use of TALENs in plants have been described and can be adapted for use in the methods described herein, see Mahfouz et al. (2011) Proc. Natl. Acad. Sci. USA, 108:2623-2628; Mahfouz (2011) GM Crops, 2:99-103; and Mohanta et al. (2017) Genes vol. 8, 12: 399). TALE nickases have also been described and can be adapted for use in methods described herein (Wu et al.; Biochem Biophys Res Commun. (2014); 446(1):261-6; Luo et al; Scientific Reports 6, Article number: 20657 (2016)).

### EMBODIMENTS

The present disclosure provides for soybean plants and plant parts comprising a mutated JAG1 gene, along with methods of making and using the same. Non-limiting embodiments of the disclosure are provided herein as follows:

(1) A soybean plant cell comprising a mutated JAG1 gene, wherein the mutated JAG1 gene encodes the polypeptide comprising the amino acid sequence of SEQ ID NO: 12, 13, 14, or an allelic variant thereof, optionally wherein the plant cell is homozygous for the mutated JAG1 gene.

(2) The soybean plant cell of embodiment 1, wherein the mutated JAG1 gene comprises: (i) the polynucleotide sequence of SEQ ID NO: 7, 8, or 9; or (ii) wherein the allelic variant of SEQ ID NO: 12, 13, or 14 is encoded by a JAG1 gene which comprises a deletion corresponding to nucleotides 426 to 430, 424 to 430, or 423 to 430 of the endogenous soybean JAG1 gene of SEQ ID NO: 1, respectively.

(3) A soybean seed comprising the soybean plant cell of embodiment 1 or 2.

(4) A soybean seed lot comprising the soybean seed of embodiment 3.

(5) The soybean seed lot of embodiment 4, wherein the seed of the seed lot are homozygous for the mutated JAG1 gene, wherein the mutated JAG1 gene encodes the polypeptide comprising the amino acid sequence of SEQ ID NO: 12 or comprises the polynucleotide sequence of SEQ ID NO: 7 and wherein the number of seeds per kilogram of seeds in the seed lot is increased in comparison to the number of seeds per kilogram of a corresponding control soybean seed lot comprising soybean seeds lacking the mutated JAG1 gene.

(6) The soybean seed lot of embodiment 5, wherein the mutated JAG1 gene encodes the polypeptide comprising the amino acid sequence of SEQ ID NO: 12 or comprises the polynucleotide sequence of SEQ ID NO: 7 and wherein the number of seeds per kilogram of seeds in the seed lot is increased by up to about 5%, 6%, 7%, 8%, 9%, 10%, 11%, or 12% in comparison to the number of seeds per kilogram of a corresponding control soybean seed lot comprising soybean seeds lacking the mutated JAG1 gene.

(7) A soybean plant comprising the soybean plant cell of embodiment 1 or 2.

(8) The soybean plant of embodiment 7, wherein the soybean plant is homozygous for the mutated JAG1 gene, wherein the mutated JAG1 gene encodes the polypeptide comprising the amino acid sequence of SEQ ID NO: 12 or comprises the polynucleotide sequence of SEQ ID NO: 7 and wherein the number of seeds per kilogram of seeds harvested from the soybean plant is increased in comparison to seeds harvested from corresponding control soybean plant lacking the mutated JAG1 gene.

(9) The soybean plant of embodiment 8, wherein the number of seeds per kilogram of seeds is increased by up to about 1%, 2%, 3%, 4%, 5%, 6%, 7%, 8%, 9%, 10%, 11%, or 12% in comparison to the number of seeds per kilogram of seeds harvested from corresponding control soybean plant lacking the mutated JAG1 gene.

(10) A soybean plant part comprising the soybean plant cell of embodiment 1 or 2, wherein said part is a leaf, stem, root, or pod.

(11) A polynucleotide comprising the sequence of SEQ ID NO: 7, 8, or 9 or encoding the polypeptide comprising the amino acid sequence of SEQ ID NO: 12, 13, or 14.

(12) A biological sample comprising the polynucleotide of embodiment 11.

(13) The biological sample of embodiment 12, wherein the sample comprises soybean meal or soybean stover.

(14) A method of producing a soybean seed lot comprising: (i) growing a population of soybean plants comprising a mutated JAG1 gene to maturity, wherein the mutated JAG1 gene encodes the polypeptide comprising the amino acid sequence of SEQ ID NO: 12, 13, 14, or an allelic variant thereof and wherein the soybean plants are homozygous for the mutated JAG1 gene; and (ii) harvesting seed from the population of soybean plants of step (i) at maturity, thereby producing the soybean seed lot.

(15) The method of embodiment 14, wherein the mutated JAG1 gene comprises: (i) the polynucleotide sequence of SEQ ID NO: 7, 8, or 9; or (ii) wherein the allelic variant of SEQ ID NO: 12, 13, or 14 comprises a deletion corresponding to nucleotides 426 to 430, 424 to 430, or 423 to 430 of the endogenous soybean JAG1 gene of SEQ ID NO: 1, respectively.

(16) The method of embodiment 14, wherein the mutated JAG1 gene encodes the polypeptide comprising the amino acid sequence of SEQ ID NO: 12 or comprises the polynucleotide sequence of SEQ ID NO: 7 and wherein the number of seeds per kilogram of seeds in the seed lot is increased in comparison to a corresponding control soybean seed lot comprising soybean seeds lacking the mutated JAG1 gene.

(17) The method of embodiment 16, wherein the number of seeds per kilogram of seeds in the seed lot is increased by up to about 5%, 6%, 7%, 8%, 9%, 10%, 11%, or 12% in comparison to the number of seeds per kilogram of control soybean seed lot comprising soybean seeds lacking the mutated JAG1 gene.

(18) A method of making a soybean plant containing a mutated JAG1 gene comprising: (a) deleting: (i) nucleotides 426 to 430 of the endogenous soybean JAG1 gene of SEQ ID NO: 1 or an allelic variant thereof in a wild-type soybean plant cell; (ii) nucleotides 424 to 430 of the endogenous soybean JAG1 gene of SEQ ID NO: 1 or an allelic variant thereof in a wild-type soybean plant cell; or (iii) nucleotides 423 to 430 of the endogenous soybean JAG1 gene of SEQ ID NO: 1 or an allelic variant thereof in a wild-type soybean plant cell; to obtain a modified soybean plant cell comprising the mutated JAG1 gene; and (b) recovering a soybean plant from the modified soybean plant cell.

(19) The method of embodiment 18, wherein: (a) the nucleotides of (i) are deleted and the mutated JAG1 gene comprises the polynucleotide sequence of SEQ ID NO: 7; (b) the nucleotides of (ii) are deleted and the mutated JAG1 gene comprises the polynucleotide sequence of SEQ ID NO: 8; or (c) the nucleotides of (iii) are deleted and the mutated JAG1 gene comprises the polynucleotide sequence of SEQ ID NO: 9.

(20) The method of embodiment 18 or 19, wherein the recovering comprises the steps of generating soybean callus from the modified soybean plant cell and generating the soybean plant from the soybean callus.

(21) The method of embodiment 18, 19, or 20, further comprising the step of harvesting seed comprising the deletion from the soybean plant.

(22) A method of producing a soybean crop comprising planting a plurality of soybean seeds of embodiment 3 or the seed lot of embodiment 4, 5, or 6.

(23) The method of embodiment 22, further comprising harvesting seed from soybean plants grown from the planted seed.

(24) The method of embodiment 23, wherein the mutated JAG1 gene encodes the polypeptide comprising the amino acid sequence of SEQ ID NO: 12 or comprises the polynucleotide sequence of SEQ ID NO: 7 and number of seeds per kilogram of harvested seeds is increased in comparison to the number of seeds per kilogram of soybean seeds harvested from corresponding control soybean plants grown from control soybean seeds lacking the mutated JAG1 gene.

(25) The method of embodiment 24, wherein the number of seeds per kilogram of harvested seed is increased by up to about 5%, 6%, 7%, 8%, 9%, 10%, 11%, or 12% in comparison to the number of seeds per kilogram of seeds harvested from corresponding control soybean plants grown from control soybean seeds lacking the mutated JAG1 gene.

(26) A method for producing a soybean by-product comprising at least one processing step of cleaning, cracking, flaking, crushing, macerating, pressing, extracting, expelling, and/or extruding the seed lot of embodiment 4, 5, or 6.

(27) The method of embodiment 26, wherein the by-product is soybean protein and wherein the soybean seed lot is subjected to processing steps comprising: (i) at least one of a cracking, flaking, crushing, pressing, and/or macerating step; (ii) extracting the cracked, flaked, crushed, pressed, and/or macerated soybean seed product from step (i) with an organic solvent to produce defatted soymeal; and (iii) extracting the defatted soymeal from step (ii) with an aqueous solvent to produce an aqueous fraction comprising soybean protein.

(28) The method of embodiment 26, wherein the by-product is soybean oil and wherein the soybean seed lot is pressed to produce the oil.

(29) The method of embodiment 26, wherein the by-product is soybean oil and wherein the soybean seed lot is subjected to processing steps comprising: (i) at least one of a cracking, flaking, crushing, pressing, and/or macerating step; and (ii) solvent extracting, expelling, and/or extruding step the cracked, flaked, crushed, pressed, and/or macerated soybean seed product from step (i) to produce the oil.

(30) A guide RNA molecule comprising a spacer RNA molecule encoded by SEQ ID NO: 2.

Additional non-limiting embodiments of the disclosure are also provided herein as follows:

1. A soybean plant cell comprising a mutated JAG1 gene, wherein the mutated JAG1 gene comprises a null mutation in the JAG1 gene encoding the polypeptide of SEQ ID NO: 10 or an allelic variant thereof and wherein the soybean plant cell lacks a loss-of-function mutation in the soybean JAG2 gene.

2. The soybean plant cell of embodiment 1, wherein the null mutation comprises a deletion of at least 1 to 16 nucleotides corresponding to nucleotides 421 to 436 of the JAG1 gene of SEQ ID NO: 1 or an allelic variant thereof.

3. The soybean plant cell of embodiment 1 or 2, wherein the mutated JAG1 gene encodes the polypeptide comprising the amino acid sequence of SEQ ID NO: 12, 13, 14, 22, 24, 26, 28, 30, or an allelic variant thereof.

4. The soybean plant cell of any one of embodiments 1 to 3, wherein the plant cell is homozygous for the mutated JAG1 gene.

5. The soybean plant cell of any one of embodiments 1 to 4, wherein the mutated JAG1 gene comprises the polynucleotide sequence of SEQ ID NO: 7, 8, 9, 15, 17, 19, 21, 23, 25, 27, 29, 31, 32, 33, 34, or 35 or an allelic variant thereof.

6. The soybean plant cell of any one of embodiments 1 to 5, wherein the soybean plant cell further comprises an A2704-12, A5547-127, BPS-CV127-9, DAS44406-6, DAS68416-4, DAS81419-2, DP305423, GTS 40-3-2, HOS, A5547-127, MON87701, MON87705, MON87708, MON87769, MON89788, MON98788, MST-FGØ72-3, or SYHTOH210 transgenic event or modification thereof; and/or wherein (ii) the soybean plant cell further comprises a null allele of the TFL1b gene.

7. A soybean seed comprising the soybean plant cell of any one of embodiments 1 to 6.

8. A soybean seed lot comprising the soybean seed of embodiment 7.

9. The soybean seed lot of embodiment 8, wherein the seed lot comprises soybean seed homozygous for the mutated JAG1 gene.

10. The soybean seed lot of embodiment 8 or 9 wherein the mutated JAG1 gene encodes the polypeptide comprising the amino acid sequence of SEQ ID NO: 12, 13, 14, 22, 24, 26, 28, 30, or an allelic variant thereof or comprises the polynucleotide sequence of SEQ ID NO: 7, 8, 9, 15, 17, 19, 21, 23, 25, 27, 29, 31, 32, 33, 34, or 35 or an allelic variant thereof.

11. The soybean seed lot of any one of embodiments 8 to 10, wherein the number of seeds per kilogram of seeds in the seed lot is increased in comparison to a corresponding control soybean seed lot comprising soybean seeds lacking the mutated JAG1 gene.

12. The soybean seed lot of any one of embodiments 8 to 10, wherein the number of seeds per kilogram of seeds in the seed lot is increased by up to about 5%, 6%, 7%, 8%, 9%, 10%, 11%, or 12% in comparison to the number of seeds per kilogram of a corresponding control soybean seed lot comprising soybean seeds lacking the mutated JAG1 gene.

13. The soybean seed lot of any one of embodiments 8 to 12, wherein the mutated JAG1 gene encodes the polypeptide comprising the amino acid sequence of SEQ ID NO: 12, 13, 14, 22, 24, 26, 28, 30, or an allelic variant thereof or comprises the polynucleotide sequence of SEQ ID NO: 7, 8, 9, 15, 17, 19, 21, 23, 25, 27, 29, 31, 32, 33, 34, or 35 or an allelic variant thereof.

14. A soybean plant comprising the soybean plant cell of any one of embodiments 1 to 6.

15. The soybean plant of embodiment 14, wherein the soybean plant is homozygous for the mutated JAG1 gene.

16. The soybean plant of embodiment 14 or 15, wherein the number of seeds per kilogram of seeds harvested from the soybean plant is increased in comparison to the number of seeds per kilogram of seeds harvested from corresponding control soybean plant lacking the mutated JAG1 gene.

17. The soybean plant of embodiment 14 or 15, wherein the number of seeds per kilogram of seeds is increased by up to about 5%, 6%, 7%, 8%, 9%, 10%, 11%, or 12% in comparison to the number of seeds per kilogram of seeds harvested from corresponding control soybean plant lacking the mutated JAG1 gene.

18. The soybean plant of any one of embodiments 14 to 17, wherein the mutated JAG1 gene encodes the polypeptide comprising the amino acid sequence of SEQ ID NO: 12, 13, 14, 22, 24, 26, 28, 30, or an allelic variant thereof or comprises the polynucleotide sequence of SEQ ID NO: 7, 8, 9, 15, 17, 19, 21, 23, 25, 27, 29, 31, 32, 33, 34, or 35 or an allelic variant thereof.

19. A soybean plant part comprising the soybean plant cell of any one of embodiments 1 to 6, wherein said part is a leaf, stem, root, or pod.

20. A polynucleotide comprising the sequence of SEQ ID NO: 7, 15, 17, 19, 21, 23, 25, 27, 29, 31, 32, 33, 34, or 35 or an allelic variant thereof or encoding the polypeptide comprising the amino acid sequence of SEQ ID NO: 12, 13, 14, 22, 24, 26, 28, 30, or an allelic variant thereof.

21. A biological sample comprising the polynucleotide of embodiment 20.

22. The biological sample of embodiment 21, wherein the sample comprises soybean meal or soybean stover.

23. A method of producing a soybean seed lot comprising: (i) growing a population of soybean plants of any one of embodiments 14 to 18 comprising a mutated JAG1 gene to maturity, wherein the soybean plants are homozygous for the mutated JAG1 gene; and (ii) harvesting seed from the population of soybean plants of step (i) at maturity, thereby producing the soybean seed lot.

24. The method of embodiment 23, wherein the mutated JAG1 gene comprises the polynucleotide sequence of SEQ ID NO: 7, 15, 17, 19, 21, 23, 25, 27, 29, 31, 32, 33, 34, or 35 or an allelic variant thereof.

25. The method of embodiment 23 or 24, wherein the number of seeds per kilogram of seeds in the seed lot is increased in comparison to the number of seeds per kilogram of a corresponding control soybean seed lot comprising soybean seeds lacking the mutated JAG1 gene.

26. The method of embodiment 23 or 24, wherein the number of seeds per kilogram of seeds in the seed lot is increased by up to about 5%, 6%, 7%, 8%, 9%, 10%, 11%, or 12% in comparison to the number of seeds per kilogram of the control soybean seed lot comprising soybean seeds lacking the mutated JAG1 gene.

27. The method of any one of embodiments 23 to 26, wherein the mutated JAG1 gene encodes the polypeptide comprising the amino acid sequence of SEQ ID NO: 12, 13, 14, 22, 24, 26, 28, 30, or an allelic variant thereof.

28. A method of making a soybean plant containing a mutated JAG1 gene comprising deleting at least 1 to 16 nucleotides corresponding to nucleotides 421 to 436 of the endogenous soybean JAG1 gene of SEQ ID NO: 1 or an allelic variant thereof in a wild-type soybean plant cell to obtain a modified soybean plant cell comprising the mutated JAG1 gene; and (b) recovering a soybean plant from the modified soybean plant cell.

29. The method of embodiment 28, wherein the mutated JAG1 gene comprises the polynucleotide sequence of SEQ ID NO: 7, 8, 9, 15, 17, 19, 21, 23, 25, 27, 29, 31, 32, 33, 34, or 35 or an allelic variant thereof.

30. The method of embodiment 28 or 29, wherein the recovering comprises the steps of generating soybean callus from the modified soybean plant cell and generating the soybean plant from the soybean callus.

31. The method of embodiment 30, further comprising the step of harvesting seed comprising the deletion from the soybean plant and/or further comprising the step of selecting for plants which lack a loss-of-function mutation in the soybean JAG2 gene.

32. A method of producing a soybean crop comprising planting the seed lot of any one of embodiments 8 to 13.

33. The method of embodiment 32, further comprising harvesting seed from soybean plants grown from the planted seed. 34 The method of embodiment 32 or 33, wherein the number of seeds per kilogram of harvested seeds is increased in comparison to the number of seeds per kilogram of soybean seeds harvested from corresponding control soybean plants grown from control soybean seeds lacking the mutated JAG1 gene. 35. The method of embodiment 32 or 33, wherein the number of seeds per kilogram of harvested seed is increased by up to about 5%, 6%, 7%, 8%, 9%, 10%, 11%, or 12% in comparison to the number of seeds per kilogram of the seeds harvested from corresponding control soybean plants grown from control soybean seeds lacking the mutated JAG1 gene.

36. A method for producing a soybean by-product comprising at least one processing step of cleaning, cracking, flaking, crushing, macerating, pressing, extracting, expelling, and/or extruding the seed lot of any one of embodiments 8 to 13.

37. The method of embodiment 36, wherein the by-product is soybean protein and wherein the soybean seed lot is subjected to processing steps comprising: (i) at least one of a cracking, flaking, crushing, pressing, and/or macerating step; (ii) extracting the cracked, flaked, crushed, pressed, and/or macerated soybean seed product from step (i) with an organic solvent to produce defatted soymeal; and (iii) extracting the defatted soymeal from step (ii) with an aqueous solvent to produce an aqueous fraction comprising soybean protein.

38. The method of embodiment 36 or 37, wherein the by-product is soybean oil and wherein the soybean seed lot is pressed to produce the oil.

39. The method of embodiment 36 or 37, wherein the by-product is soybean oil and wherein the soybean seed lot is subjected to processing steps comprising: (i) at least one of a cracking, flaking, crushing, pressing, and/or macerating step; and (ii) solvent extracting, expelling, and/or extruding step the cracked, flaked, crushed, pressed, and/or macerated soybean seed product from step (i) to produce the oil.

40. A guide RNA molecule comprising a Cas12 direct repeat element which is operably linked to the spacer RNA molecule encoded by SEQ ID NO: 2 EXAMPLES

### Example 1. Generation of mJAG1 Soybean

The plasmid pIN1340 was created to transform soybean plants and disrupt the open reading frame of the JAGGED1 (Jag1) gene (Glyma.20g116200) (SEQ ID NO: 1) through CRISPR-mediated gene editing. This plasmid was constructed using the strategy and techniques described by Cermik et al., 2017, The Plant Cell. 29 (6) 1196-1217; DOI: 10.1105/tpc.16.00922). The pIN1340 vector has the following two functional expression cassettes between the right and left T-DNA border. A Solanum lycopersicum ubiquitin gene promoter and 5′ untranslated region (UTR) drives the expression a CRISPR-Cas nuclease transcript. The Cas gene had a SV40 nuclear localization signal (NLS) fused to the 5′ end and a nucleoplasmin NLS fused to the 3′ end. The Cas coding sequence with the NLS fusions was codon optimized for soy expression as set forth in WO2021202397. The coding sequence was followed by an Arabidopsis thaliana heat shock gene terminator. Another expression cassette is made up of an Arabidopsis thaliana U6-26 promoter driving the expression of a CRISPR guide RNA comprising a crRNA fused to the RNA encoded by the JAG1_g3 (SEQ ID NO: 2) spacer designed to target the Glycine max JAG1 gene and followed by an RNA polymerase iii termination signal.

The other functional elements of the pIN1340 vector were derived from a standard Agrobacterium binary transformation plasmid that can replicate in both Escherichia coli and Agrobacterium tumefaciens. They are a T-DNA right border sequence (SEQ ID NO: 4) followed by an expression cassette to confer glyphosate resistance to the transgenic plants. This cassette consisted of the Arabidopsis thaliana Ubiquitin 10 gene promoter and 5′ UTR, Agrobacterium sp. strain CP4 5-enolpyruvylshikimate-3-phosphate synthase (EPSPS) gene and the Pisum sativum rib-1,5-bisphospate carboxylase (rbcS) small subunit gene terminator. The insertion site was followed by a T-DNA left border sequence (SEQ ID NO: 3).

The plasmid pIN1340 was transformed into Agrobacterium tumefaciens EHA105 (Hood et al., 1993, Transgenic Research. 2: 208-218. doi:10.1007/BF01977351) by electroporation following standard techniques. Frozen glycerol stocks were prepared for use in plant transformation.

Transgenic TO soybean events were made by Agrobacterium-mediated transformation with vector pIN1340. Sterilized soybean seeds were imbibed in water overnight, and explants were prepared as mature cotyledon halves with trimmed hypocotyls. The explants went through the typical transformation and regeneration steps of infection and co-cultivation, shoot induction and elongation and selection, rooting, and transplanting to soil to produce T1 seeds (see, for example, Li et al, Optimization of Agrobacterium-Mediated Transformation in Soybean (2017) Frontiers in Plant Science v8 Article 246; Pareddy et al. Transgenic Res. 2020 June; 29(3):267-281. doi: 10.1007/s11248-020-00198-8).

T1 progeny of TO plants were grown and genotyped by AmpSeq. T1 plants with the following edits set forth in Table 2, homozygous or heterozygous as indicated in the following example, or segregating wild types, were selected for phenotyping.

TABLE 2
Gene Edits

| Description | SEQ ID NO |
|---|---|
| Wild type JAG1 DNA fragment (annotated) | 5 |
| Wild type JAG1 peptide fragment encoded by | 6 |
| SEQ ID NO: 5 | |
| 1:5D mJAG1 deletion | 7 |
| −2:7D mJAG1 deletion | 8 |
| −3:8D mJAG1 deletion | 9 |
| 1:5D mJAG1 deletion translation product | 12 |
| −2:7D mJAG1 deletion translation product | 13 |
| −3:8D mJAG1 deletion translation product | 14 |

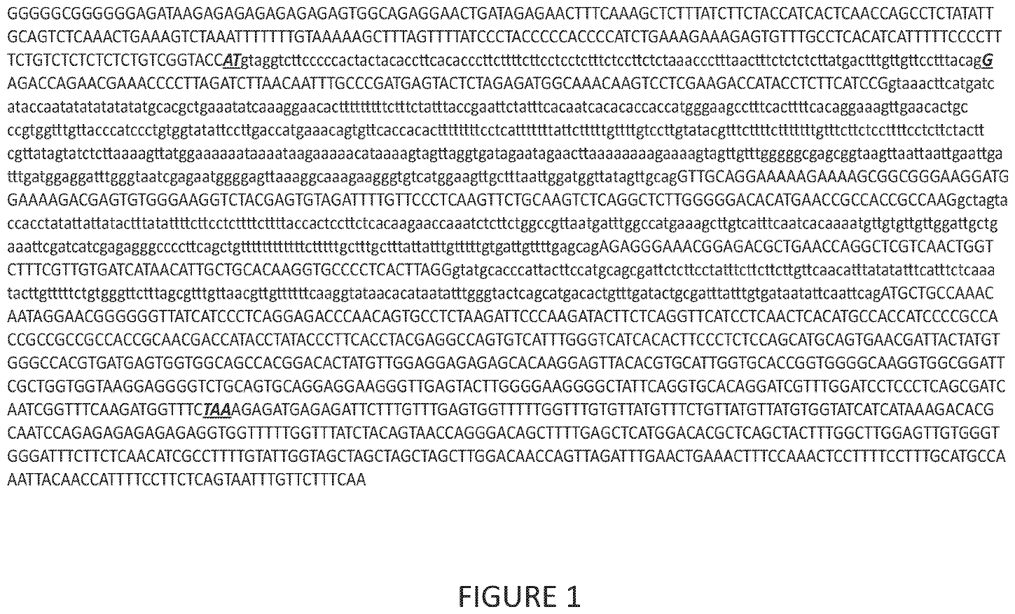

The reference wild-type JAG1 DNA sequence is depicted in FIG. 1 and mJAG1 deletion sequences are depicted in FIG. 2. The three deletions produce frameshift mutations which result in the polypeptides of SEQ ID NO: 12, 13, and 14. The GmJag2 locus was sequenced as well, but no edits were found.

### Example 2. Performance of mJAG1 Soybean

T1 progeny plants with the genotypes noted in the Table 3 below were grown out on a field at a sparse density, and phenotyped for seed count per plant, number of seeds per pod, total seed weight (grams) per plant, and single seed weight (seed weight/seed count).

TABLE 3
Analysis of T1 Progeny plants

| | JAG1 | | | | Single |
|---|---|---|---|---|---|
| Plant ID | Genotype | SEEDCT | NSPP | SEEDWT | SEEDWT |
| NINF1170.005 | WT | 523 | 2.43 | 93.42 | 0.179 |
| NINF1170.006 | WT | 580 | 2.57 | 106.45 | 0.184 |
| NINF1170.009 | WT | 592 | 2.55 | 112.14 | 0.189 |
| NINF1170.010 | WT | 549 | 2.41 | 99.55 | 0.181 |
| T1GM00071CB.pIN1304.520.025 | WT | 107 | 1.88 | 21.37 | 0.200 |
| T1GM00073CB.pIN1304.440.007 | WT | 582 | 2.56 | 107.92 | 0.185 |
| T1GM00073CB.pIN1304.440.012 | WT | 526 | 2.44 | 92.65 | 0.176 |
| T1GM00073CB.pIN1304.440.014 | WT | 674 | 2.75 | 127.92 | 0.190 |
| T1GM00073CB.pIN1304.440.015 | WT | 594 | 2.55 | 112.97 | 0.190 |
| T1GM00075CB.pIN1338.533.005 | WT | 472 | 2.40 | 91.67 | 0.194 |
| T1GM00075CB.pIN1338.533.009 | WT | 678 | 2.36 | 119.28 | 0.176 |
| T1GM00075CB.pIN1338.533.010 | WT | 636 | 2.48 | 113.38 | 0.178 |
| T1GM00075CB.pIN1338.533.011 | WT | 546 | 2.36 | 91.51 | 0.168 |
| T1GM00075CB.pIN1338.533.012 | WT | 403 | 2.53 | 61.58 | 0.153 |
| T1GM00075CB.pIN1338.533.013 | WT | 423 | 2.53 | 61.41 | 0.145 |
| T1GM00075CB.pIN1338.533.014 | WT | 417 | 2.40 | 70.1 | 0.168 |
| T1GM00075CB.pIN1338.533.015 | WT | 455 | 2.42 | 76.64 | 0.168 |
| T1GM00075CB.pIN1340.300.002 | 1:5D/1:5D | 375 | 2.42 | 64.96 | 0.173 |
| T1GM00075CB.pIN1340.300.049 | 1:5D/1:5D | 574 | 2.47 | 87.02 | 0.152 |
| T1GM00075CB.pIN1340.300.052 | 1:5D/1:5D | 519 | 2.40 | 83.89 | 0.162 |
| T1GM00075CB.pIN1340.300.053 | 1:5D/1:5D | 471 | 2.60 | 73.94 | 0.157 |
| T1GM00075CB.pIN1340.300.058 | 1:5D/1:5D | 542 | 2.40 | 89.52 | 0.165 |
| T1GM00075CB.pIN1340.307.007 | WT | 529 | 2.44 | 96.32 | 0.182 |
| T1GM00075CB.pIN1340.308.003 | −3:8D/−3:8D | 525 | 2.64 | 79.08 | 0.151 |
| T1GM00075CB.pIN1340.308.020 | −3:8D/−3:8D | 473 | 2.53 | 75.49 | 0.160 |
| T1GM00075CB.pIN1340.308.026 | −3:8D/−3:8D | 509 | 2.33 | 90.46 | 0.178 |
| T1GM00075CB.pIN1340.308.030 | −3:8D/−3:8D | 409 | 2.43 | 69.26 | 0.169 |
| T1GMIW000100.045.001 | WT | 407 | 2.45 | 67.7 | 0.166 |
| T1GMIW000100.064.013 | WT | 281 | 2.34 | 52.65 | 0.187 |
| T1GMIW000100.064.015 | WT | 221 | 2.13 | 43.65 | 0.198 |
| T1GMIW000100.064.026 | WT | 383 | 2.29 | 69.36 | 0.181 |
| T1GMIW000100.064.047 | WT | 459 | 2.37 | 94.58 | 0.206 |
| T1GMIW000100.064.050 | WT | 506 | 2.64 | 97.98 | 0.194 |
| T1GMIW000100.064.071 | −2:7D/−2:7D | 489 | 2.29 | 89.38 | 0.183 |

The data indicates that the score seed phenotypes are not significantly impacted by the mJAG1 alleles.

T1 progeny plants with the genotypes noted in Table 4 below were grown out on a field at a sparse density, and phenotyped for leaf length and width. Leaf measurements were taken at the 5^{th} node about 6 weeks after planting, and at the 8^{th} node about 7 weeks after planting.

TABLE 4
Leaf Phenotypes

| | JAG1 | 5^{th} | 5th | 5th | 8^{th} | 8th | 8^{th} |
|---|---|---|---|---|---|---|---|
| Plant ID | Genotype | L_cm | W_cm | L/W | L_cm | W_cm | L/W |
| NINF1170.005 | WT | 9.4 | 6.8 | 1.38 | 12.7 | 8.7 | 1.46 |
| NINF1170.006 | WT | 9.5 | 6.6 | 1.44 | 11.4 | 7.6 | 1.50 |
| NINF1170.009 | WT | 9.5 | 6.7 | 1.42 | 12.3 | 8.6 | 1.43 |
| NINF1170.010 | WT | 9.6 | 6.7 | 1.43 | 11 | 7.6 | 1.45 |
| T1GM00075CB.pIN1338.533.005 | WT | 7.3 | 5.5 | 1.33 | 10.3 | 6 | 1.72 |
| T1GM00075CB.pIN1338.533.009 | WT | 8 | 5.6 | 1.43 | 11.1 | 7.3 | 1.52 |
| T1GM00075CB.pIN1338.533.010 | WT | 9.3 | 6.1 | 1.52 | 12 | 7.5 | 1.60 |
| T1GM00075CB.pIN1338.533.011 | WT | 8.8 | 6 | 1.47 | 10.9 | 7.9 | 1.38 |
| T1GM00075CB.pIN1338.533.012 | WT | 8.4 | 5.9 | 1.42 | 9.7 | 5.6 | 1.73 |
| T1GM00075CB.pIN1338.533.013 | WT | 8.6 | 5.8 | 1.48 | 10.2 | 6 | 1.70 |
| T1GM00075CB.pIN1338.533.014 | WT | 7.5 | 5.3 | 1.42 | 9 | 5.4 | 1.67 |
| T1GM00075CB.pIN1338.533.015 | WT | 7.5 | 5.5 | 1.36 | 9 | 5.8 | 1.55 |
| T1GM00075CB.pIN1340.300.002 | 1:5D/1:5D | 10 | 4.8 | 2.08 | 13.5 | 6 | 2.25 |
| T1GM00075CB.pIN1340.300.049 | 1:5D/1:5D | 11.3 | 5.6 | 2.02 | 14 | 7 | 2.00 |
| T1GM00075CB.pIN1340.300.052 | 1:5D/1:5D | 9.4 | 5 | 1.88 | 13.1 | 6.5 | 2.02 |
| T1GM00075CB.pIN1340.300.053 | 1:5D/1:5D | 10.5 | 5.5 | 1.91 | 13.5 | 6.4 | 2.11 |
| T1GM00075CB.pIN1340.300.058 | 1:5D/1:5D | 10.3 | 5 | 2.06 | 13.1 | 6.5 | 2.02 |
| T1GM00075CB.pIN1340.307.007 | WT | 10.2 | 6.3 | 1.62 | 13.2 | 7.6 | 1.74 |
| T1GM00075CB.pIN1340.308.003 | −3:8D/−3:8D | 9.8 | 4.6 | 2.13 | 14.8 | 6.8 | 2.18 |
| T1GM00075CB.pIN1340.308.020 | −3:8D/−3:8D | 9.9 | 4.8 | 2.06 | 13.6 | 5.7 | 2.39 |
| T1GM00075CB.pIN1340.308.026 | −3:8D/−3:8D | 10.1 | 5 | 2.02 | 13.7 | 7 | 1.96 |
| T1GM00075CB.pIN1340.308.030 | −3:8D/−3:8D | 9.5 | 4.7 | 2.02 | 13.3 | 5.3 | 2.51 |
| T1GMIW000100.045.001 | WT | 7.6 | 5.2 | 1.46 | 9.7 | 6.5 | 1.49 |
| T1GMIW000100.064.013 | WT | 9 | 6 | 1.50 | 11 | 6.4 | 1.72 |
| T1GMIW000100.064.015 | WT | 8 | 5 | 1.60 | | | |
| T1GMIW000100.064.026 | WT | 7.1 | 5.1 | 1.39 | 10.1 | 5.4 | 1.87 |
| T1GMIW000100.064.047 | WT | 9.4 | 6.4 | 1.47 | 10.7 | 7.2 | 1.49 |
| T1GMIW000100.064.050 | WT | 8.7 | 6 | 1.45 | 11.5 | 7.3 | 1.58 |
| T1GMIW000100.064.071 | −2:7D/−2:7D | 10.1 | 5.1 | 1.98 | 12.2 | 6.3 | 1.94 |

The data in Table 4 indicates that the leaves of the plants homozygous for the mJAG1 mutations are narrower and elongated.

### Example 3. Seed Size Distribution in mJAG1 Soybean

Seed size distribution was measured using screens with round holes ranging from 3.6 ( 9/64 inches) to 8. 7 mm (22/64 inches) in diameter at intervals of either 0.4 or 0.8 mm (Egli et al, Agronomy Journal, Volume 79, Issue 3, 463-467 1987). The screens were stacked on top of each other each sample was placed on the top screen.

For each of the sieves, the seed that remained at the top of each screen was weighed. The distribution of seed sizes is shown in Table 5 below, with the numbers indicating the weight (g) of the portion of seeds that remained on the circular screen of indicated size (in inches) from a 1000 g subsample shaken for 5 minutes across 9 screens ordered in decreasing size. Collected for 1 replicate at 6 locations.

TABLE 5
Seed size

| | GENOTYPE | | | | |
|---|---|---|---|---|---|
| | | | | JAG1 | Unedited |
| | 1:5D/1:5D | −3:8D/−3:8D | −2:7D/−2:7D | Null | WT CTRL |
| Size Screening: | 0.0 | 0.0 | 0.0 | 0.0 | 0.0 |
| 22/64 Round Screen | | | | | |
| Size Screening: | 0.0 | 0.0 | 0.0 | 0.0 | 0.0 |
| 20/64 Round Screen | | | | | |
| Size Screening: | 5.9 | 10.6 | 9.6 | 5.3 | 10.1 |
| 18/64 Round Screen | | | | | |
| Size Screening: | 488.2 | 484.9 | 477.5 | 490.7 | 499.3 |
| 16/64 Round Screen | | | | | |
| Size Screening: | 485.1 | 487.9 | 489.7 | 485.3 | 473.4 |
| 14/64 Round Screen | | | | | |
| Size Screening: | 17.3 | 14.0 | 19.6 | 15.7 | 14.5 |
| 13/64 Round Screen | | | | | |
| Size Screening: | 3.2 | 2.2 | 3.2 | 2.6 | 2.5 |
| 12/64 Round Screen | | | | | |
| Size Screening: | 0.1 | 0.1 | 0.1 | 0.1 | 0.1 |
| 11/64 Round Screen | | | | | |
| Size Screening: | 0.0 | 0.0 | 0.0 | 0.0 | 0.0 |
| 10/64 Round Screen | | | | | |

These results show that seeds from the 1:5D/1:5D homozygous mJAG1 plants are not of a significantly smaller size than those from the wild type controls.

### Example 4. Seed Weights and Yields of mJAG1 Soybean Grown in Different Locations

Plants homozygous for the indicated mJAG1 alleles and in the same genetic background were grown at various locations, as indicated in Table 6 below. From most locations, five subsamples were collected from three replicated plots and the seeds were weighed and counted. Avg. 1000-Swt is the weight of one thousand seeds in grams. The yield per plant or yield per plot values were largely similar for all these mutants and wild type in these trials.

TABLE 6
Seed weights and Yields

| | | Allele | | | |
|---|---|---|---|---|---|
| Location | | 1:5D | −2:7D | −3:8D | WT |
| Iowa 1 | Avg. 1000-Swt | 150.1 | 152.6 | 153.7 | 156.7 |
| | Std. dev. of 1000-Swt | | 3.3 | 3.4 | 3.4 |
| | Avg. Seed/lb | 3,022.0 | 2,972.7 | 2,952.3 | 2,895.2 |
| | Std. dev. of Seed/lb | | 64.7 | 63.5 | 62.8 |
| Iowa 2 | Avg. 1000-Swt | 156.4 | 151.0 | 152.6 | 156.4 |
| | Std. dev. of 1000-Swt | | 4.0 | 3.1 | 3.3 |
| | Avg. Seed/lb | 2,899.0 | 3,006.7 | 2,973.3 | 2,901.2 |
| | Std. dev. of Seed/lb | | 80.0 | 62.3 | 62.7 |
| Ohio 1 | Avg. 1000-Swt | 152.8 | 163.4 | 164.5 | 167.3 |
| | Std. dev. of 1000-Swt | | 2.5 | 2.4 | 7.2 |
| | Avg. Seed/lb | 2,969.0 | 2,777.5 | 2,758.0 | 2,715.0 |
| | Std. dev. of Seed/lb | | 41.7 | 39.4 | 114.2 |
| Illinois | Avg. 1000-Swt | 144.8 | 148.3 | 150.2 | 150.5 |
| | Std. dev. of 1000-Swt | | 2.1 | 2.6 | 3.0 |
| | Avg. Seed/lb | 3,133.0 | 3,058.0 | 3,019.7 | 3,014.6 |
| | Std. dev. of Seed/lb | | 43.9 | 53.0 | 60.1 |
| Indiana 1 | Avg. 1000-Swt | 119.1 | 124.6 | 127.0 | 127.2 |
| | Std. dev. of 1000-Swt | 0.8 | 2.0 | 3.6 | 2.6 |
| | Avg. Seed/lb | 3,810.0 | 3,639.3 | 3,574.3 | 3,568.0 |
| | Std. dev. of Seed/lb | 25.5 | 59.5 | 103.0 | 72.1 |
| Puerto | Avg. 1000-Swt | 143.8 | | 141.2 | 144.5 |
| Rico 1 | Std. dev. of 1000-Swt | 3.3 | | 15.0 | 14.3 |
| | Avg. Seed/lb | 3,156.2 | | 3,242.4 | 3,164.9 |
| | Std. dev. of Seed/lb | 72.6 | | 312.0 | 288.4 |
| Puerto | Avg. 1000-Swt | 145.9 | | 152.1 | 149.3 |
| Rico 2 | Std. dev. of 1000-Swt | 1.0 | | 4.0 | 1.9 |
| | Avg. Seed/lb | 3,104.5 | | 2,979.5 | 3,035.0 |
| | Std. dev. of Seed/lb | 20.0 | | 77.2 | 40.2 |
| Nebraska | Avg. 1000-Swt | 158.3 | 157.2 | 156.3 | 160.5 |
| | Std. dev. of 1000-Swt | | 2.0 | 5.1 | 2.0 |
| | Avg. Seed/lb | 2,864.0 | 2,885.7 | 2,903.3 | 2,826.8 |
| | Std. dev. of Seed/lb | | 35.5 | 95.4 | 35.9 |
| Indiana 2 | Avg. 1000-Swt | 149.5 | | 154.0 | 168.0 |
| | Std. dev. of 1000-Swt | 7.8 | | | 10.7 |
| | Avg. Seed/lb | 3,034.0 | | 2,943.0 | 2,710.0 |
| | Std. dev. of Seed/lb | 158.4 | | | 167.1 |
| Green | Avg. 1000-Swt | 161.8 | | 164.5 | 183.3 |
| house | Std. dev. of 1000-Swt | 8.0 | | 11.6 | 4.3 |
| | Avg. Seed/lb | 2,808.8 | | 2,767.8 | 2,476.3 |
| | Std. dev. of Seed/lb | 137.6 | | 196.2 | 58.2 |

These results indicate a consistently lower and tighter distribution of weight of seeds from plants homozygous for the mJAG1 1:5D allele.

### Example 5. Generation of Additional mJAG1 Mutations

Additional mJAG1 mutant soybean plants were generated essentially as described in Example 1. T1 progeny of T0 plants were grown and genotyped by AmpSeq. T1 and later generation plants that were homozygous the following JAG1 gene edits set forth in Table 7 were selected for phenotyping.

TABLE 7
Additional mJAG1 mutants

| | SEQ ID NO | |
|---|---|---|
| | OF mJAG1 DNA | SEQ ID NO OF |
| | FRAGMENT OR | GENOMIC DNA |
| | ENCODED mJAG1 | WITH mJAG1 |
| Description | PROTEIN | DELETION |
| −2:10D mJAG1 deletion | 21 | 31 |
| −2:10D mJAG1 deletion | 22 | |
| translation product | | |
| −2:5D mJAG1 deletion | 23 | 32 |
| −2:5D mJAG1 deletion | 24 | |
| translation product | | |
| 2:1D mJAG1 deletion | 25 | 33 |
| 2:1D mJAG1 deletion | 26 | |
| translation product | | |
| 1:4D mJAG1 deletion | 27 | 34 |
| 1:4D mJAG1 deletion | 28 | |
| translation product | | |
| 1:7D mJAG1 deletion | 29 | 35 |
| 1:7D mJAG1 deletion | 30 | |
| translation product | | |
| Large mJAG1 Deletion | A deletion comprising at | |
| | least nucleotides 421 to | |
| | 436 of SEQ ID NO: 1. | |
| Large Deletion translation | Not Determined | |
| product | | |

### Example 6. Seeds Per Pound of Seed for mJAG1 Soybean in 2022 Field Tests in Comparison to Checks

Plants homozygous for the indicated mJAG1 alleles and in the same genetic background were field tested in the summer of 2022 in the central United States. The number of seeds per pound of harvested seed for each mJAG1 allele was determined and compared to checks which lack the mJAG1 allele but were otherwise isogenic as indicated in Table 8 below. The number of seeds per pound of seed was increased for all mJAG1 alleles relative to checks in for the NING1295, TEND2128, TENF2132, and TENG2141T genotypes. The TENF2147L genotype with the 1:4D and 1:7D mJAG1 alleles exhibited a decrease in seed number per pound.

TABLE 8
Calculated Seeds/lb (from individual seed weight avg)

| | | | Avg | | | |
|---|---|---|---|---|---|---|
| | | | for | | | |
| | Allele (SEQ ID | | mJAG1 | Null | Check | % |
| Genotype | NO) | Plants | allele | Avg | Avg | Check |
| NING1295 | −2:10D (SEQ ID | 36 | 3204 | 2937 | 3080 | 104% |
| | NO: 21) | | | | | |
| | −2:5D (SEQ ID | 33 | 3228 | 2937 | 3080 | 104.8% |
| | NO: 23) | | | | | |
| | −2:7D (SEQ ID | 34 | 3125 | 2937 | 3080 | 101.4% |
| | NO: 8) | | | | | |
| | 2:1D (SEQ ID NO: | 34 | 3174 | 2937 | 3080 | 103% |
| | 25) | | | | | |
| TEND2128 | 1:5D (SEQ ID NO: | 29 | 2863 | 2904 | 2779 | 103% |
| | 7) | | | | | |
| | −3:8D (SEQ ID | 31 | 2811 | 2904 | 2779 | 101% |
| | NO: 9) | | | | | |
| TENF2132 | −2:5D (SEQ ID | 26 | 2941 | NA | 2792 | 105% |
| | NO: 23) | | | | | |
| | Large Deletion | 34 | 3086 | NA | 2792 | 110.5% |
| | (ND) | | | | | |
| TENF2147L | 1:4D (SEQ ID NO: | 34 | 2894 | | 3061 | 94.5% |
| | 27) | | | | | |
| | 1:7D (SEQ ID NO: | 40 | 2864 | | 3061 | 93.5% |
| | 29) | | | | | |
| TENG2141T | −2:5D (SEQ ID | 42 | 3014 | | 2891 | 104% |
| | NO: 23) | | | | | |

### Example 7. Summary of Biological Sequences Provided in Sequence Listing

The following Table 9 describes biological sequences provided herein.

TABLE 9
Biological Sequences

| | SEQ | | |
|---|---|---|---|
| | ID | SEQUENCE | |
| SEQUENCE DESCRIPTION | NO | TYPE | SOURCE |
| Wild type JAG1 genomic DNA | 1 | DNA | Glycine max |
| DNA encoding JAG1_g3 spacer | 2 | DNA | synthetic |
| | | | construct |
| T-DNA left border | 3 | DNA | synthetic |
| | | | construct |
| T-DNA right border | 4 | DNA | synthetic |
| | | | construct |
| Wild type JAG1 DNA fragment | 5 | DNA | Glycine max |
| (annotated) | | | |
| Wild type JAG1 peptide fragment | 6 | PRT | Glycine max |
| encoded by SEQ ID NO: 5 | | | |
| 1:5D mJAG1 deletion DNA | 7 | DNA | synthetic |
| fragment | | | construct |
| −2:7D mJAG1 deletion DNA | 8 | DNA | synthetic |
| fragment | | | construct |
| −3:8D mJAG1 deletion DNA | 9 | DNA | synthetic |
| fragment | | | construct |
| Wild-type JAG1 | 10 | PRT | Glycine max |
| Wild-type JAG1 coding sequence | 11 | DNA | Glycine max |
| (CDS) | | | |
| 1:5D mJAG1 deletion translation | 12 | PRT | synthetic |
| product (protein) | | | construct |
| −2:7D mJAG1 deletion translation | 13 | PRT | synthetic |
| product (protein) | | | construct |
| −3:8D mJAG1 deletion translation | 14 | PRT | synthetic |
| product (protein) | | | construct |
| 1:5D mJAG1 deletion genomic | 15 | DNA | synthetic |
| sequence | | | construct |
| Predicted mJAG1 1:5D CDS | 16 | DNA | synthetic |
| | | | construct |
| −2:7D mJAG1 deletion genomic | 17 | DNA | synthetic |
| sequence | | | construct |
| −2:7D mJAG1CDS (DNA) | 18 | DNA | synthetic |
| | | | construct |
| −3:8D mJAG1 deletion genomic | 19 | DNA | synthetic |
| sequence | | | construct |
| 3:8D mJAG1 CDS (DNA) | 20 | DNA | synthetic |
| | | | construct |
| minus 2:10D mJAG1 deletion | 21 | DNA | synthetic |
| DNA fragment | | | construct |
| minus 2:10D mJAG1 deletion | 22 | PRT | synthetic |
| translation product | | | construct |
| minus 2:5D mJAG1 deletion DNA | 23 | DNA | synthetic |
| fragment | | | construct |
| minus 2:5D mJAG1 deletion | 24 | PRT | synthetic |
| translation product | | | construct |
| 2:1D mJAG1 deletion DNA | 25 | DNA | synthetic |
| fragment | | | construct |
| 2:1D mJAG1 deletion translation | 26 | PRT | synthetic |
| product | | | construct |
| 1:4D mJAG1 deletion DNA | 27 | DNA | synthetic |
| fragment | | | construct |
| 1:4D mJAG1 deletion translation | 28 | PRT | synthetic |
| product | | | construct |
| 1:7D mJAG1 deletion DNA | 29 | DNA | synthetic |
| fragment | | | construct |
| 1:7D mJAG1 deletion translation | 30 | PRT | synthetic |
| product | | | construct |
| minus 2:10D mJAG1 deletion | 31 | DNA | synthetic |
| genomic DNA | | | construct |
| minus 2:5D mJAG1 deletion | 32 | DNA | synthetic |
| genomic DNA | | | construct |
| 2:1D mJAG1 deletion genomic | 33 | DNA | synthetic |
| DNA | | | construct |
| 1:4D mJAG1 deletion genomic | 34 | DNA | synthetic |
| DNA | | | construct |
| 1:7D mJAG1 deletion genomic | 35 | DNA | synthetic |
| DNA | | | construct |
| mJAG2 wild-type (WT) genomic | 36 | DNA | Glycine max |
| DNA | | | |
| mJAG2 WT CDS | 37 | DNA | Glycine max |
| mJAG2 WT protein | 38 | PRT | Glycine max |
| TFL1b WT genomic DNA | 39 | DNA | Glycine max |
| TFL1b WT CDS | 40 | DNA | Glycine max |
| TFL1b WT protein | 41 | PRT | Glycine max |

All cited patents and patent publications referred to in this application are incorporated herein by reference in their entirety. All of the materials and methods disclosed and claimed herein can be made and used without undue experimentation as instructed by the above disclosure and illustrated by the examples. Although the materials and methods of this disclosure have been described in terms of embodiments and illustrative examples, it will be apparent to those of skill in the art that substitutions and variations can be applied to the materials and methods described herein without departing from the concept, spirit, and scope of the disclosure. All such similar substitutes and modifications apparent to those skilled in the art are deemed to be within the spirit, scope, and concept of the disclosure as encompassed by the embodiments of the disclosures recited herein and the specification and appended claims.

What is claimed is:

1.-19. (canceled)

20. A polynucleotide comprising the sequence of SEQ ID NO: 7, 15, 17, 19, 21, 23, 25, 27, 29, 31, 32, 33, 34, or 35 or an allelic variant thereof or encoding the polypeptide comprising the amino acid sequence of SEQ ID NO: 12, 13, 14, 22, 24, 26, 28, 30, or an allelic variant thereof.

21. A biological sample comprising the polynucleotide of claim 20.

22. The biological sample of claim 21, wherein the sample comprises soybean meal or soybean stover.

23. The biological sample of claim 22, wherein the soybean seed meal is defatted.

24. The biological sample of claim 22, wherein the soybean seed meal is non-defatted.

25.-39. (canceled)

40. A guide RNA molecule comprising a Cas12 direct repeat element which is operably linked to the spacer RNA molecule encoded by SEQ ID NO: 2.
